(19) United States
(12) Patent Application Publication
(10) Pub. No.: US 2026/0076743 A1
(43) Pub. Date: Mar. 19, 2026

(54) METHOD FOR PROVIDING SPINAL SURGERY IMAGES AND A RECORDING MEDIUM RECORDING A COMPUTER-READABLE PROGRAM FOR EXECUTING THE METHOD

(71) Applicants: Youmin BAE, Seoul (KR); Seungmin BAE, Seoul (KR)

(72) Inventor: Junseok BAE, Seoul (KR)

(21) Appl. No.: 19/314,639

(22) Filed: Aug. 29, 2025

(30) Foreign Application Priority Data
Sep. 19, 2024 (KR) 10-2024-0126505
May 12, 2025 (KR) 10-2025-0061563

(51) Int. Cl. A61B 34/10; A61B 90/00; G06F 3/0482; G06T 7/73; G06T 19/00; G16H 20/40; G16H 40/63

(52) U.S. Cl. CPC A61B 34/10; A61B 90/37; G06T 7/75; G06T 19/006; G16H 20/40; G16H 40/63; A61B 2034/104; A61B 2034/105; A61B 2034/107; A61B 2090/365; A61B 2090/367; A61B 2090/373; A61B 2090/374; A61B 2090/3762; G06F 3/0482; G06T 2200/24; G06T 2207/10068; G06T 2207/10081; G06T 2207/10088; G06T 2207/20021; G06T 2207/30004; G06T 2210/41

(57) ABSTRACT

Provided is a spinal surgery image-providing method and a computer-readable recording medium storing a program for executing the method. The image-providing method includes: a step of storing a three-dimensional model generated using first image data acquired by a first medical imaging device when a patient is in a first posture; a step of storing second image data acquired by a second medical imaging device when the patient is in a second posture; a three-dimensional model adjustment step of adjusting the three-dimensional model using the second image data; an insertion path computation step of computing an optimal surgical instrument insertion path toward a surgical target; and an insertion path display step of displaying the optimal surgical instrument insertion path on a screen of an augmented reality device.

### CROSS-REFERENCE TO RELATED APPLICATIONS

This application claims priority to Korean Patent Application Nos. 10-2024-0126505 and 10-2025-0061563, filed on Sep. 19, 2024, and May 12, 2025, respectively, in the Korean Intellectual Property Office, the disclosure of which is incorporated herein by reference.

### TECHNICAL FIELD

The present invention relates to a recording medium storing a computer-readable program for executing a method of providing spinal surgery images, and more particularly, to a method for enabling effective spinal surgery by eliminating errors that may occur between a three-dimensional (3D) model of a patient and the actual state of the patient, through the use of an augmented reality (AR) device or projection mapping device in spinal surgery or the like.

### BACKGROUND ART

In general, minimally invasive spinal surgery proceeds through the following process. The necessity of surgery is assessed through a patient's prior medical records, imaging scans (CT, MRI), and physical examinations. The surgical site and necessary procedures are planned, and anesthesia methods are determined depending on the patient's condition and surgical site. At the beginning of the surgery, the site is sterilized thoroughly and the patient is positioned appropriately, usually in the prone or lateral position. A skin incision of the minimum necessary size is made, and tools such as a trocar are used to separate muscle and tissue to secure the pathway. A tubular dilator is inserted to gradually enlarge the approach path. An endoscope or surgical microscope is inserted into the dilator to visualize the surgical site, and forceps, cutters, lasers, or radiofrequency probes are inserted to remove discs or bone. Herniated discs are excised, or the spinal canal is widened to relieve nerve compression. If necessary, pedicle screws or other fixation devices are inserted to stabilize the spine. Finally, the incision site is sutured to complete the procedure.

However, such conventional minimally invasive spinal surgery has the drawback that it proceeds based on limited information regarding internal body structures, relying heavily on the surgeon's tactile sense. Furthermore, in the process of guiding surgical instruments into the intended target location, there is a risk of damaging critical organs (e.g., central nerves). There is also a risk that, under incomplete information, the surgeon may finish the operation without sufficiently removing the target lesion, or may damage surrounding organs in addition to the target tissue. Recently, attempts have been made to apply augmented reality (AR) devices in surgery, but practical application has been difficult due to discrepancies between the 3D model and the actual state of the patient.

### DETAILED DESCRIPTION

### Technical Problem to be Solved

An embodiment of the present invention is intended to provide a method for providing spinal surgery images and a recording medium storing a computer-readable program for executing the method, which eliminates errors between a patient's 3D model and the actual state of the patient when using an augmented reality device or a projection mapping device in spinal surgery, thereby enabling effective spinal surgery.

It should be understood that the problems described above are not exhaustive. Other problems not mentioned will be apparent to those skilled in the art from the following description.

### Solution to the Problem

To achieve the foregoing objectives, a spinal surgery image-providing method according to an embodiment of the present invention includes:
storing a 3D model generated using first image data obtained by a first medical imaging device when the patient is in a first posture;
storing second image data obtained by a second medical imaging device when the patient is in a second posture;
adjusting the 3D model using the second image data in a 3D model adjustment step;
computing an optimal insertion path of a surgical instrument toward the surgical target in an insertion-path computation step; and
displaying the computed optimal insertion path of the surgical instrument on the screen of an augmented reality device or on the patient's body surface in an insertion-path display step.

Furthermore, the 3D model adjustment step may include: extracting feature points from the second image data; extracting corresponding points from the 3D model; mapping the correspondence between the feature points and the corresponding points; estimating the second posture using the correspondence; and adjusting the 3D model according to the estimated second posture.

Additionally, the 3D model may include one or more layers, and such layers may include at least one selected from a skin layer, bone layer, intervertebral disc layer, ligament layer, nerve layer, and vascular layer. The method may further include a range-setting step of defining the range of the 3D model to be displayed on the screen of the augmented reality device or on the patient's body surface. The range-setting step may include: displaying, on the screen of the augmented reality device or an interface system, a first list of predefined anatomical structures and receiving a selection input from the user; or displaying the 3D model on the screen of the augmented reality device or interface system and receiving a selection input from the user; or displaying, on the screen of the augmented reality device or interface system, a second list of layers and receiving a selection input from the user.

Additionally, the first medical imaging device may be a CT (Computed Tomography) device or an MRI (Magnetic Resonance Imaging) device, and the 3D model may include at least one of the skin layer, bone layer, intervertebral disc layer, ligament layer, nerve layer, and vascular layer. The second medical imaging device may be a C-arm device.

Furthermore, the 3D model adjustment step may include: displaying the 3D model on a screen of the augmented reality device or an interface system; receiving, with respect to a characteristic anatomical structure of the 3D model, a first reference point input by a user in a first reference point reception step; displaying the second image data visually on the screen of the augmented reality device or the interface system; receiving, with respect to a characteristic anatomical structure of the second image data, a second reference point input by a user in a second reference point reception step; and precisely adjusting the 3D model using the first and second reference points in a fine adjustment step.

The characteristic anatomical structure may include at least one of a tip of a spinous process or an iliac crest.

In addition, the second reference point reception step may include: visually displaying, on the screen of the augmented reality device or the interface system, second-1 image data obtained from a top-down view of the patient, and receiving a second-1 reference point input by the user with respect to the characteristic anatomical structure in the second-1 image data; and visually displaying, on the screen of the augmented reality device or the interface system, second-2 image data obtained from a lateral view of the patient, and receiving a second-2 reference point input by the user with respect to the characteristic anatomical structure in the second-2 image data.

The fine adjustment step may include: mapping the correspondence between the first and second reference points; estimating the second posture based on the mapped correspondence; and adjusting the 3D model according to the estimated second posture.

The insertion-path computation step may further include a surgical target setting step, in which a surgical target is designated on the 3D model. The surgical target setting step may include: invoking, via the augmented reality device or interface system, a surgical target setting menu and storing as a surgical target model an item selected by the user from the menu; or storing, as a surgical target model, a region designated by the user on the displayed 3D model.

The insertion-path computation step may also include: dividing the 3D model into a plurality of small voxels; storing the initial position from which a surgical instrument is to be inserted and the target position to be reached; assigning movement costs to each voxel in a cost-assignment step; and calculating costs required to pass through voxels between the initial position and the target position, thereby determining, as the optimal insertion path, the path with the lowest cost.

In the cost-assignment step, relatively high costs may be assigned to voxels adjacent to predefined anatomical structures, or to voxels located in positions into which the surgical instrument cannot be inserted or is difficult to insert.

The method may further include a surgical instrument model display step, in which a surgical instrument model representing the surgical instrument in 3D is displayed on the screen of the augmented reality device or on the surface of the patient's body.

The surgical instrument model display step may include: detecting information regarding the surgical instrument from the second image data or receiving information regarding the surgical instrument; and displaying the surgical instrument model based on the information on the surgical instrument, wherein the surgical instrument model is displayed overlapped with the 3D model.

In addition, on the screen of the augmented reality device or the patient's body surface, the optimal surgical instrument insertion path and the surgical instrument model may be displayed overlapping together.

The method may further include, after the insertion-path display step, visually displaying, on the screen of the augmented reality device or the interface system, third image data obtained by a third medical imaging device. The third medical imaging device may be an endoscopic camera.

The method may further include a surgical state-reflection step of displaying, on at least one of the screen of the augmented reality device, the patient's body surface, or the interface system, the 3D model reflecting the surgical state. The surgical state-reflection step may include: computing surgical progress using at least one of the number of times the surgical instrument model has passed through a location, the three-dimensional range traversed by the surgical instrument model, and the residence time of the surgical instrument model at each location; and reflecting, on the surgical target model, a region in which surgery has been performed according to the surgical progress.

The surgical state-reflection step may further include: displaying the surgical target model with different colors according to the surgical progress; or displaying the surgical target model with varying transparency according to the surgical progress; and providing a notification to the user via the augmented reality device when the surgical instrument model approaches within a predetermined distance of a predefined anatomical structure or when the surgical progress reaches a preset level.

### Effects of the Invention

According to the prior art, it was difficult for the surgeon to determine the exact position of the surgical instrument. According to an embodiment of the present invention, the optimal surgical instrument insertion path and the surgical instrument model can be displayed overlapped in the same display area. Thus, the surgeon can accurately determine the position of the surgical instrument by viewing the instrument model displayed on the screen of the augmented reality device or on the surface of the patient's body, and at the same time, the optimal insertion path is displayed, enabling safe and effective insertion of the surgical instrument.

In the prior art, the surgeon typically performed spinal surgery while simultaneously viewing a C-arm planar image (a top-down image of the patient's back) and a lateral image (a side view of the patient's torso). Accordingly, it was difficult to intuitively determine the three-dimensional position and angle of the instrument inserted into the body. In contrast, according to the present invention, the user can easily and intuitively determine the current three-dimensional position of the surgical instrument. By simultaneously viewing the instrument model and the 3D model, the surgeon can guide the instrument safely and accurately toward the surgical target.

According to the prior art, the surgeon has relied on tactile sense to perform disc-removal surgery. Since an endoscope displays only an enlarged portion, it is difficult to precisely determine which part of the target has been removed. Consequently, in some cases, too much disc material (or nucleus pulposus) is removed, or insufficient disc material is removed. According to the present invention, the ranges of removed and remaining disc material can be visually and clearly identified, enabling more accurate surgery.

The effects of the present invention are not limited to those described above, and various additional effects are included within the present specification.

### BRIEF DESCRIPTION OF THE DRAWINGS

FIG. 1 is a diagram illustrating a spinal surgery image-providing system according to an embodiment of the present invention.

FIGS. 2 and 3 are diagrams briefly illustrating the operation process of the system of FIG. 1.

FIG. 4 is a diagram showing the basic configuration of a spinal surgery image-providing system according to another embodiment of the present invention.

FIG. 5 is a diagram showing an application state of the spinal surgery image-providing system of FIG. 4.

FIGS. 6A to 6C are flowcharts for explaining a spinal surgery image-providing method according to an embodiment of the present invention.

FIG. 7 is a diagram illustrating a display-range setting step.

FIGS. 8A to 8D are diagrams explaining a process for setting reference points on the 3D model and the second image.

FIG. 9 is a diagram illustrating a fifth display area.

FIGS. 10A and 10B are diagrams illustrating an insertion-path computation step for determining the optimal surgical instrument insertion path.

FIG. 11 is a diagram illustrating the optimal surgical instrument insertion path displayed in a sixth display area.

FIGS. 12A and 12B are diagrams illustrating the display screen of a third image obtained by a third medical imaging device.

FIG. 13 is a diagram illustrating a projection image area and the optimal surgical instrument insertion path in an embodiment employing projection mapping.

FIGS. 14A, 14B, and 15 are diagrams illustrating the procedure of endoscopic discectomy.

FIG. 16 is a diagram illustrating the process of reducing a protruding nucleus pulposus using forceps.

FIGS. 17A and 17B are diagrams illustrating the state of a surgical target reflected in 3D in real time and displayed in an eighth display area.

FIG. 18 is a block diagram of a spinal surgery image-providing system according to an embodiment of the present invention.

### DETAILED DESCRIPTION OF THE EMBODIMENTS

Hereinafter, embodiments of the present invention will be described in detail with reference to the drawings.

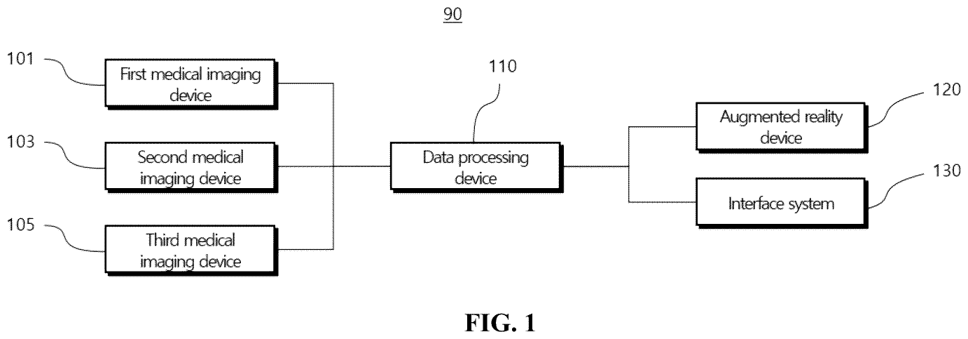

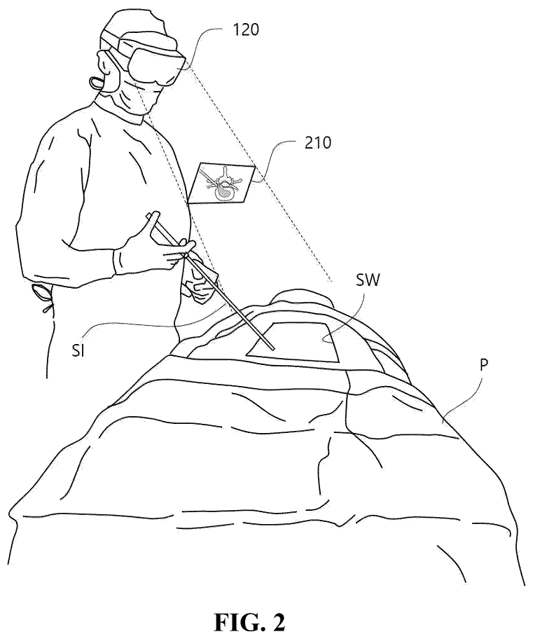

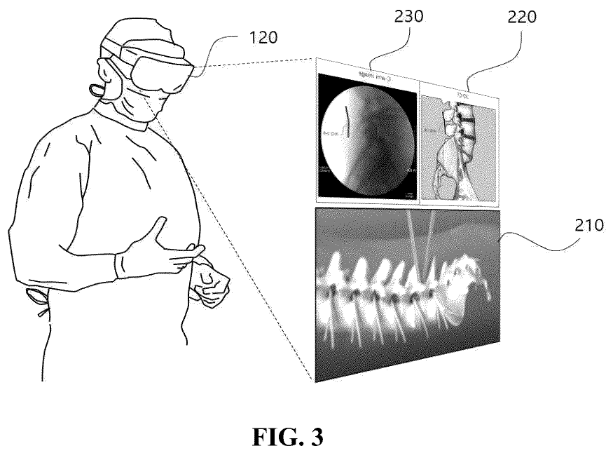

FIG. 1 is a diagram illustrating a spinal surgery image-providing system according to an embodiment of the present invention. FIGS. 2 and 3 are diagrams briefly explaining the operation process of the system of FIG. 1.

As shown in FIG. 1, a spinal surgery image-providing system 90 according to an embodiment of the invention includes a plurality of medical imaging devices (101, 103, 105), a data processing device 110 such as a server or computer, an augmented reality device 120, and some or all of an interface system 130.

The phrase “some or all” means that the spinal surgery image-providing system 90 may be implemented even if certain components such as the interface system 130 are omitted, or that certain or all components of the data processing device 110 may be integrated into the augmented reality device 120. For convenience of understanding the invention, however, the system is described as including all of the components.

The plurality of medical imaging devices 101, 103, 105 may include various types of devices usable for implementing the spinal surgery image-providing method according to embodiments of the present invention. These medical imaging devices include a first medical imaging device 101, a second medical imaging device 103, and a third medical imaging device 105.

The first medical imaging device 101 may include 3D CT equipment or 3D MRI equipment. The first medical imaging device 101 may capture the inside of the patient's body and generate first image data. A three-dimensional model 301 of the patient's body can then be generated based on the first image data.

For example, the first image data may be tomographic image data, and using the first medical imaging device 101 or other devices, a 3D model may be constructed from the data.

The second medical imaging device 103 may be a C-arm, which generates second image data. A C-arm is a special imaging device that can capture real-time X-ray images of bones and joints. The second image data is obtained by capturing the patient's body using the C-arm. This second image data may be displayed as a second image on the augmented reality device 120.

The posture of the patient when captured by the second medical imaging device 103 may differ from the posture when captured by the first medical imaging device 101. For example, the first image data may be acquired when the patient is lying supine facing upward, whereas the second image data may be obtained when the patient is prone or lying laterally.

The third medical imaging device 105 may be an endoscopic camera, which can be directly inserted into the body to capture internal images.

The data processing device 110 may include a server or the like. The server may be a computer or a cloud server remotely connected through a communication network. In embodiments, the data processing device 110 may be a computer or server installed at a hospital where minimally invasive spinal surgery is performed. The data processing device 110 communicates with the first, second, and third medical imaging devices (101, 103, 105), the augmented reality device 120, and the interface system 130 through wired or wireless communication to transmit and receive data. The data processing device 110 performs major computation and information processing operations.

For example, the data processing device 110, together with the augmented reality device 120, may execute the steps of the spinal surgery image-providing method according to embodiments of the invention. Details will be described later in connection with the spinal surgery image-providing method.

The data processing device 110 may control the augmented reality device 120 to allow a user to view the patient's 3D model, or alternatively, a control unit within the augmented reality device 120 may manage the display process.

The augmented reality device 120, which may be worn as a headset or glasses, is a wearable device that enables the user to view both real-world images and artificially generated images simultaneously. The device combines sensors and display technology to allow users to superimpose immersive digital content onto their everyday environment.

According to an embodiment, the augmented reality device 120 may include core components such as a transparent display, gaze-tracking sensors, cameras, an onboard processor, and a communication module. The transparent display allows the user wearing the AR headset to see the real world while viewing digital images overlaid thereon, using technologies such as waveguides, transparent OLED, or transparent LCD. Some AR devices project digital images directly onto lenses using micro-projectors.

The gaze-tracking sensors may include gyroscopes, accelerometers, and magnetic sensors to track head movements and position, thereby maintaining precise alignment between real-world imagery and digital content. The cameras may include forward-facing cameras and depth sensors to recognize the surrounding environment and perform 3D mapping, enabling spatial awareness, gesture recognition, and object recognition. The onboard processor may include CPUs, MPUs, or GPUs, and the computing unit within the AR headset handles tasks such as graphics processing, sensor data integration, and user interface operations. The communication module may employ Wi-Fi or Bluetooth to exchange data with other devices, particularly the data processing device 110.

The interface system 130 may be a device installed in the operating room for displaying medical images. Data and information necessary for surgical staff may be displayed on monitors or similar displays. For example, when the data processing device 110 is a computer, the interface system 130 may serve as the display device connected thereto, showing medical images or other useful data.

In addition to the foregoing, the medical imaging devices 101, 103, 105, the data processing device 110, the augmented reality device 120, and the interface system 130 of FIG. 1 may perform various operations, and related details will be described in further detail below.

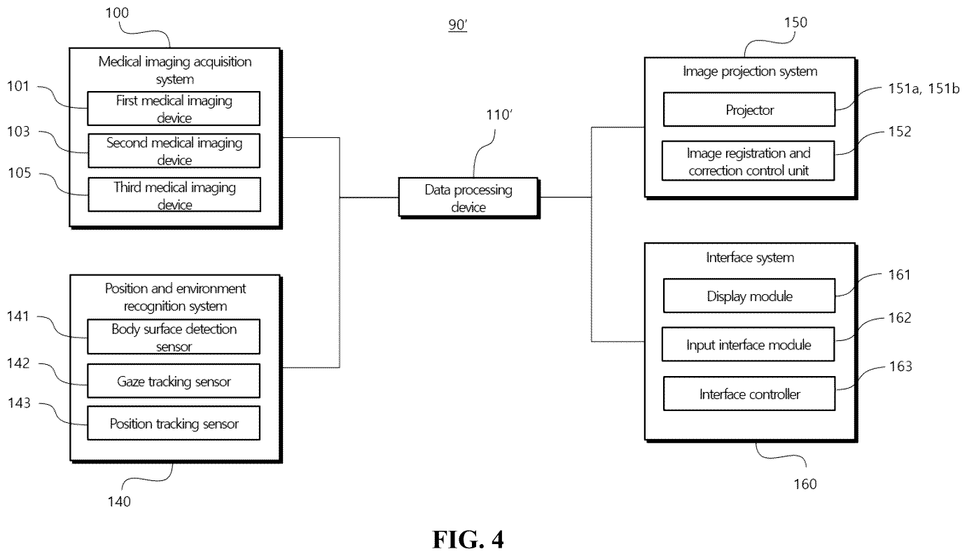

In addition, although not shown in FIG. 1, a position-tracking sensor may also be included. A position-tracking sensor tracks, in real time, the location and orientation of the surgical instrument (SI) during surgery to ensure registration with the 3D model image. This enables the user to intuitively confirm the precise position of the surgical instrument inside the body, and to accurately overlay the patient's anatomical structure and the instrument movement onto the 3D model, thereby enhancing surgical precision. A detailed explanation of the position-tracking sensor will be given with reference to FIG. 4.

As shown in FIG. 2, the augmented reality device (120) may display, in virtual space, a first display area (210) (also referred to as a main window). The first display area (210) corresponds to the region in the augmented reality in which images are displayed when the augmented reality device (120) is worn by the user, and may display a three-dimensional model of the patient (P). Although represented as a rectangular window in FIG. 2, the configuration is not limited thereto. When the augmented reality device (120) is worn, the entire visible field may be regarded as the main window. To the user (surgeon), the actual appearance of the patient and the three-dimensional model in the main window may be overlapped and displayed. The user wearing the augmented reality device (120) inserts the surgical instrument (SI) into the skin exposed within the surgical window (SW) region. The surgical window (SW, also referred to as a surgical field) refers to the region where only the surgical site is exposed while the remainder of the body is covered with drapes.

As shown in FIG. 3, the screen of the augmented reality device (120) may further display auxiliary display areas (220, 230) (also referred to as reference-point setting windows) in addition to the first display area (210). The first display area (210) is a portion of the augmented reality screen and may display the three-dimensional model generated using the first image data. The actual appearance of the patient and the three-dimensional model may be overlapped and displayed together. The auxiliary display areas (220, 230) are virtual display areas used for adjusting the three-dimensional model to match the actual state of the patient. While wearing the augmented reality device (120), the user may turn the auxiliary display areas (220, 230) on or off using hand gestures, voice commands, or a controller. The auxiliary display areas (220, 230) may include a second display area (220), in which the three-dimensional model is displayed, and a third display area (230), in which second image data (such as C-arm images) is displayed.

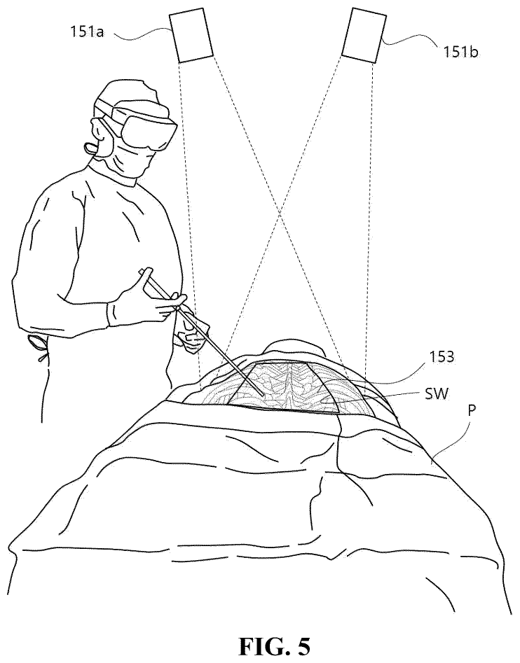

FIG. 4 is a diagram illustrating the basic configuration of a spinal surgery image-providing system according to another embodiment of the present invention. FIG. 5 is a diagram illustrating an application state of the spinal surgery image-providing system of FIG. 4. FIGS. 4 and 5 illustrate an embodiment employing projection mapping or spatial augmented reality (SAR).

As shown in FIG. 4, the spinal surgery image-providing system (90′) according to another embodiment includes a medical imaging acquisition system (100), a position and environment recognition system (140), an image projection system (150), an interface system (160), and a data processing device (110′).

The medical imaging acquisition system (100) may include the plurality of medical imaging devices (101, 103, 105) described in the embodiment of FIGS. 1 to 3. That is, the medical imaging acquisition system (100) may include the first medical imaging device (101), the second medical imaging device (103), and the third medical imaging device (105). The first medical imaging device (101) may include three-dimensional CT equipment or three-dimensional MRI equipment. The second medical imaging device (103) may include a C-arm. The third medical imaging device (105) may be an endoscopic camera that can be inserted directly into the patient's body to capture internal images. Image data acquired from the first medical imaging device (101), the second medical imaging device (103), and the third medical imaging device (105) may be transmitted to the image registration and correction control unit (152) or to the data processing device (110′) for processing. Descriptions of overlapping technical components with the embodiment of FIGS. 1 to 3 are omitted.

The position and environment recognition system (140) detects and tracks in real time the patient's body surface, surgical instruments, the user's gaze, and the surrounding environment. The position and environment recognition system (140) includes a body surface detection sensor (141), a gaze-tracking sensor (142), and a position-tracking sensor (143).

The body surface detection sensor (141) detects in real time the curvature of the patient's body surface and collects the corresponding data. To perform three-dimensional scanning of the patient's body surface, the body surface detection sensor (141) may include LiDAR, stereo cameras (depth cameras), or infrared sensors (IR sensors).

The gaze-tracking sensor (142) detects the gaze direction of the user. When projection mapping is applied, if the user's viewpoint is fixed, an effective three-dimensional perception may be provided. However, if the user's viewpoint changes, the projected three-dimensional image may be distorted. By detecting the user's gaze and adjusting the visual perspective of the projected three-dimensional image, a natural stereoscopic effect can be provided as if the user were actually viewing the internal structure. The gaze-tracking sensor (142) may include an optical-based gaze-tracking system or an inertial measurement unit (IMU)-based head-tracking system. The optical-based gaze-tracking system may employ infrared cameras or RGB-D cameras to detect the user's head movement and gaze direction in real time. The IMU-based head-tracking system may include wearable devices such as smart glasses or lightweight headsets incorporating IMU sensors (gyroscope, accelerometer, magnetic sensor) to detect head movement.

The position-tracking sensor (143) tracks in real time the position and orientation of the surgical instrument during surgery so that it is registered with the three-dimensional model image. Through this, the user can intuitively confirm the exact position of the surgical instrument inside the body, and the patient's anatomical structure and the movement of the surgical instrument can be accurately overlaid on the three-dimensional model, thereby improving surgical precision.

The position-tracking sensor (143) may use an optical tracking method, which is a type of optical position-tracking technology, employing infrared cameras and markers (reflective markers or LED markers) to track the position of surgical instruments and the patient. In this method, optical markers are attached to surgical instruments and the patient's body surface, and three-dimensional coordinates are calculated by triangulation using two or more infrared cameras, enabling six degrees of freedom (6DoF: X, Y, Z+pitch, roll, yaw) tracking.

The position-tracking sensor (143) may alternatively employ a magnetic tracking method, which uses a magnetic field to track the position of surgical instruments in real time within the body. In this method, a magnetic field transmitter coil emits a magnetic field, and a receiver coil attached to the surgical instrument measures the field strength, which is then used to calculate the three-dimensional position of the surgical instrument. The optical tracking method and the magnetic tracking method may be used together.

The position and environment recognition system (140) may further include a control unit for controlling the body surface detection sensor (141), the gaze-tracking sensor (142), and the position-tracking sensor (143), and such a control unit may be implemented as a component of the data processing device (110′).

The image projection system (150) projects a three-dimensional model image onto the surface of the patient's body so that the user can intuitively recognize the internal anatomical structure and the position of the surgical instrument. The image projection system (150) may be based on projection mapping technology or spatial augmented reality (SAR) technology, and performs functions of aligning the three-dimensional model image with the patient's actual anatomical structure and projecting it in real time without distortion. By doing so, the user can check the projected three-dimensional model directly on the patient's body without looking at a monitor, and thus the status of the surgical instrument can be recognized more accurately and intuitively.

The image projection system (150) may include projectors (151a, 151b) and the image registration and correction control unit (152). The projectors (151a, 151b) are devices that project the three-dimensional model image onto the patient's body surface. The projectors (151a, 151b) project images to form a projection image area (153) on the body surface of the patient, in which the three-dimensional model image is displayed. The projectors (151a, 151b) may be designed to project precise images even in a narrow surgical environment. It is preferable to employ laser-based DLP or LCD projectors supporting high contrast ratios and high resolution. Although FIG. 5 illustrates two projectors (151a, 151b), the number is not limited thereto. Depending on the curvature of the patient's body, the viewing angle of the user, and shadows cast by surgical instruments, two or more projectors may be arranged from multiple directions.

The image registration and correction control unit (152) aligns the three-dimensional model image with the actual anatomical structure of the patient and automatically corrects distortions. In this embodiment, the image registration and correction control unit (152) is described as being included in the image projection system (150), but it may alternatively be implemented as part of the data processing device (110′).

Three-dimensional body surface data collected by the body surface detection sensor (141) may be transmitted to the image registration and correction control unit (152), which automatically deforms the projected image shape in consideration of the curvature of the body surface. For example, if the body surface is curved, the projected image is pre-distorted so that it appears normal to the user. If the patient's posture changes slightly during surgery, the body surface detection sensor (141) detects the change and updates the surface state image in real time. Based on the scanned data, projection mapping techniques can be applied to correct distortion. Even if the patient moves slightly during surgery, automatic correction may be performed so that the projected image remains aligned with the patient's body.

The process of extracting the three-dimensional shape of the body using the body surface detection sensor (141) may be performed by employing existing algorithms such as Point Cloud Processing or Depth Map Extraction. The process of automatically deforming the shape of the projection image in consideration of the curvature of the body surface may be performed by employing existing algorithms such as Homography Transformation, Thin Plate Spline (TPS) Warping, or Bezier Surface Fitting. If the patient's posture changes slightly during surgery, the body surface detection sensor (141) detects the change and updates the body surface data in real time.

The image registration and correction control unit (152) receives data from the gaze-tracking sensor (142) and determines the head movement and gaze direction of the user. The image registration and correction control unit (152) also determines the body part of the patient that the user is actually viewing.

The image registration and correction control unit (152) adjusts the perspective of the projected image according to the gaze direction of the user, and when the user moves to a different angle, the projected three-dimensional image responds immediately so that a natural stereoscopic effect is maintained. In the real-time perspective adjustment step according to the user's gaze, a Head-Tracking & Dynamic Perspective Correction algorithm may be applied. This algorithm calculates the observation angle of the user using the gaze-tracking sensor (142), and reflects the result to adjust in real time the perspective of the projected image. In the real-time perspective adjustment step according to the user's gaze, an Optical Flow Estimation algorithm may also be applied. This algorithm analyzes the pixel displacement between an existing frame and a new frame when the user's gaze changes, and performs correction based on the analysis.

The image registration and correction control unit (152) may receive data from the position-tracking sensor (143) and perform a step of computing the three-dimensional position and orientation of the surgical instrument, and a step of registering the position of the surgical instrument with the three-dimensional model and correcting errors. Accordingly, the exact position of the surgical instrument inside the body may be represented in the three-dimensional model from the viewpoint of the user.

In the step of computing the three-dimensional position and orientation of the surgical instrument, the three-dimensional position and orientation of the surgical instrument may be computed based on data provided by the position-tracking sensor (143), such as optical position tracking or magnetic position tracking. Such a step may be performed by applying algorithms such as an Extended Kalman Filter (EKF) or a Particle Filter. In the step of registering the position of the surgical instrument with the three-dimensional model, algorithms such as the Iterative Closest Point (ICP) algorithm, a landmark-based registration algorithm, or a feature-based Random Sample Consensus (RANSAC) algorithm may be applied. In the step of correcting errors in the position of the surgical instrument, algorithms such as an Adaptive Kalman Filter, a non-rigid deformation correction algorithm, or an AI-powered registration correction algorithm may be applied.

The interface system (160) is a system that supports the user in intuitively manipulating and controlling the three-dimensional model image, the projection image, and the surgical instrument tracking information. The interface system (160) may allow contact-type or non-contact-type operation in the surgical environment using a keyboard, a touchscreen, gesture recognition, or voice commands. The interface system (160) according to an embodiment of the present invention may include a display module (161), an input interface module (162), and an interface controller (163).

The display module (161) is a device that displays medical images during surgery. The input interface module (162) is a device that receives input information from the user through methods such as a keyboard, a touchscreen, gesture recognition, or voice commands. In the case of a touchscreen method, the display module (161) and the input interface module (162) may be configured as a single module. The interface controller (163) is a computing device that analyzes the input data and performs manipulation of the three-dimensional model image and the projection image. Although the interface controller (163) is described as a component included in the interface system (160), the interface controller (163) may alternatively be implemented as a component included in the data processing device (110′).

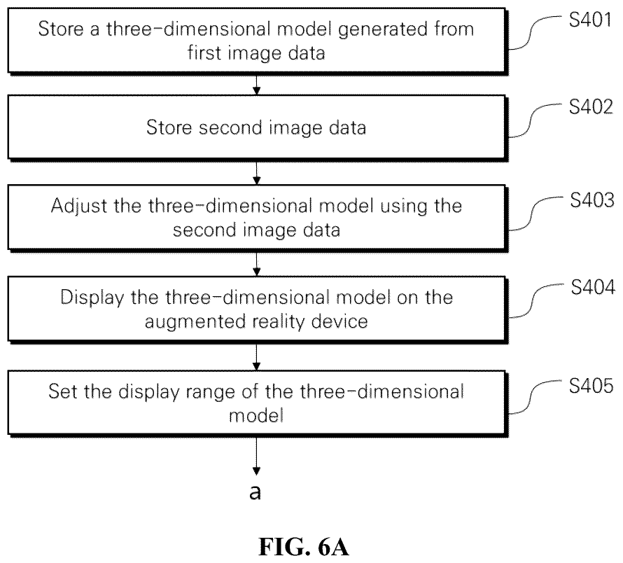

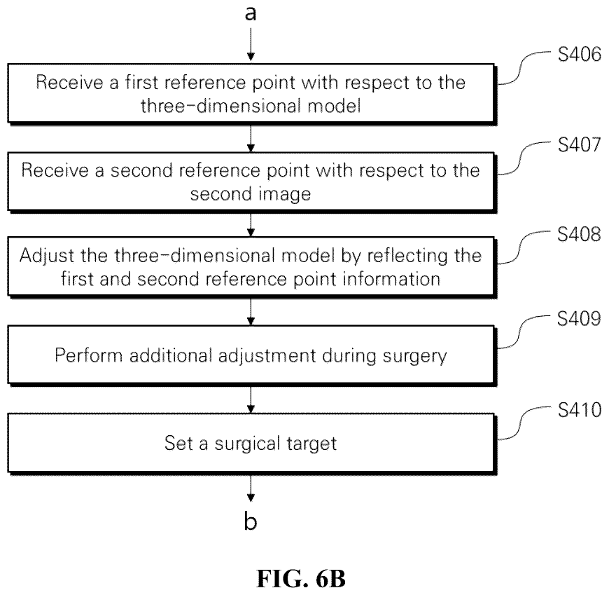

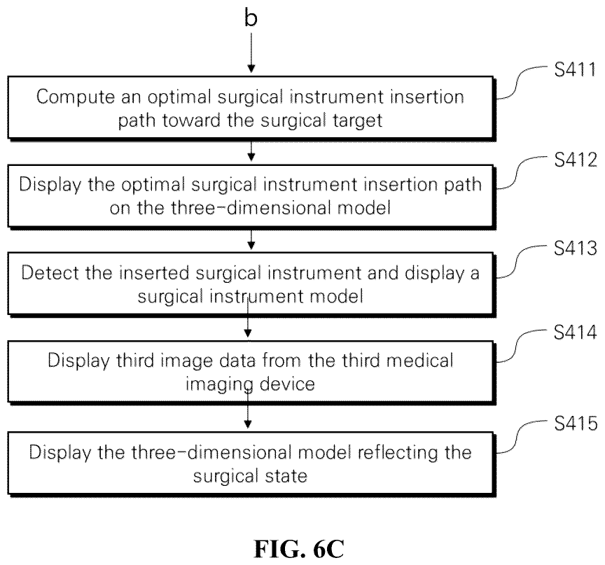

FIGS. 6A to 6C are flowcharts for explaining a spinal surgery image-providing method according to an embodiment of the present invention. In FIGS. 6A to 6C, “a” and “b” indicate connection points that are linked to each other.

### <Step S401>

Referring to FIG. 1 and FIGS. 6A to 6C, step S401 will be described. When the patient is in a first posture, a step (S401) of storing a three-dimensional model generated using first image data acquired by the first medical imaging device (101) may be performed. Step S401 may be performed by at least one of the data processing device (110), the augmented reality device (120), or another computing device connected via wired or wireless communication means. The same applies to the remaining steps (S402 to S415) described below.

The first posture may be a posture in which the patient is lying down facing the ceiling. The first image data is data acquired by photographing the internal structure of the patient's body using three-dimensional CT equipment or MRI equipment. For convenience of description, an embodiment in which three-dimensional CT data is acquired using three-dimensional CT equipment will be described. The CT equipment scans the body of the patient from multiple angles using X-rays, and generates a detailed three-dimensional image from this data. This provides precise images of internal organs, bones, and tissues, and is useful for diagnosing diseases or detecting abnormalities.

Based on the first image data, a three-dimensional model of the patient's internal body structure may be constructed using a computer program. The step of constructing the three-dimensional model may be performed by the first medical imaging device (101), the data processing device (110), the augmented reality device (120), or another computing device connected via wired or wireless communication means.

A general three-dimensional CT (Computed Tomography) device is equipped with a function of generating a three-dimensional model of the internal structure of a patient. This function is achieved by acquiring a plurality of tomographic images (slices) and reconstructing them into three dimensions through computer software. The three-dimensional model is a stereoscopic model constructed using the first image data. The first image data may be three-dimensional CT data or three-dimensional MRI data. The three-dimensional model may include a plurality of layers. That is, the plurality of layers may include a skin layer of the patient, a bone layer, an intervertebral disc layer, a ligament layer, a nerve layer, and a vascular layer.

In an embodiment based on a projection mapping method, a step (S401′) similar to step S401 may also be performed. Referring to FIG. 4, FIG. 5, and FIGS. 6A to 6C, step S401′ may be performed by at least one of the medical imaging acquisition system (100), the data processing device (110′), or another computing device connected via wired or wireless communication means. Descriptions of technical steps overlapping with the embodiment of FIGS. 1 to 3 are omitted.

### <Step S402>

When the patient is in a second posture, a step (S402) of storing second image data acquired by the second medical imaging device (103) may be performed. The second posture may be a posture in which the patient is lying prone or a posture in which the patient is lying on the side. The second medical imaging device (103) may be a C-arm, which generates the second image data.

The second image data may be X-ray image data captured using the C-arm equipment. Generally, CT scanning is performed when the patient is lying supine, and thereafter, the patient is transferred to the operating room. During patient transfer, the posture of the patient may change. In addition, in the case of performing endoscopic discectomy, the surgery is performed while the patient is lying prone. Accordingly, the three-dimensional model generated based on the CT image data (or the first image data) may differ from the actual state of the patient. In the following description, a case of performing endoscopic discectomy will be assumed.

In the operating room, while the patient is in a prone posture, the C-arm equipment is used to acquire a frontal X-ray image (for example, an image obtained from above viewing the patient's back downward) and a lateral X-ray image (for example, an image obtained from the side viewing the patient's flank). Preferably, X-ray images from various directions such as the anterior, posterior, and lateral directions are acquired in order to improve accuracy.

The C-arm is X-ray equipment used in operating rooms, mainly for providing real-time X-ray images to monitor surgical situations. The equipment is named for its C-shaped arc, and it can move around the patient's body at various angles.

In an embodiment based on a projection mapping method, a step (S402′) similar to step S402 may also be performed. Referring to FIG. 4, FIG. 5, and FIGS. 6A to 6C, step S402′ may be performed by at least one of the medical imaging acquisition system (100), the data processing device (110′), or another computing device connected via wired or wireless communication means. Descriptions of technical steps overlapping with the embodiment of FIGS. 1 to 3 are omitted.

### <Step S403>

Next, a three-dimensional model adjustment step (S403) of adjusting the three-dimensional model using the second image data may be performed. Since CT scanning is generally performed while the patient is lying supine, the body shape differs from that of the prone posture. Therefore, the three-dimensional model is adjusted using the C-arm image data (i.e., the second image data) acquired while the patient is currently in the prone posture. This means that the posture of the three-dimensional model is adjusted to match the actual surgical posture of the patient.

The three-dimensional model adjustment step (S403) may include a step (S403-1) of extracting feature points from the second image data, a step (S403-2) of extracting corresponding points from the three-dimensional model that correspond to the feature points, a step (S403-3) of mapping a correspondence between the feature points and the corresponding points, a step (S403-4) of estimating the second posture using the correspondence, and a step (S403-5) of adjusting the three-dimensional model according to the estimated second posture.

More specifically, the step (S403-1) of extracting feature points from the second image data may be a step of extracting feature points (particularly specific points of bone structures) from the X-ray images. For this purpose, a deep learning-based point detector or an image processing algorithm may be used. After the step (S403-2) of extracting corresponding points from the three-dimensional model that correspond to the feature points, the step (S403-3) of mapping the correspondence between the feature points and the corresponding points may be performed.

Accordingly, points appearing in the X-ray image may be connected with points of the three-dimensional model. Then, the step (S403-4) of estimating the second posture using the correspondence may be performed. As a result, it may be determined which posture the patient is taking (i.e., prone posture).

As pose estimation algorithms, various algorithms such as Iterative Closest Point (ICP), Go-ICP (Global Optimal ICP), Generalized ICP (GICP), Coherent Point Drift (CPD), Normal Distributions Transform (NDT), Fast Global Registration (FGR), Non-Rigid Registration, Thin Plate Splines (TPS), B-Splines Free-Form Deformations (FFD), Demons Algorithm, Large Deformation Diffeomorphic Metric Mapping (LDDMM), Optical Flow, and Symmetric Normalization (SyN) may be applied.

Subsequently, a step (S403-5) of adjusting the three-dimensional model according to the estimated second posture may be performed. In this step, the skeletal structure of the existing three-dimensional model is adjusted into the prone posture determined during the posture estimation step. This adjustment is achieved through deformation of the skeletal structure, and the surface (mesh) of the model is also deformed accordingly. The surface deformation process is a process of naturally deforming the surface of the three-dimensional model according to the deformation of the skeletal structure. For this purpose, techniques such as blend shapes or skin clustering may be used. The surface of the model may be finely adjusted to reflect deformations of the human body caused by pressure applied in the prone posture.

In an embodiment based on a projection mapping method, a step (S403′) similar to step S403 may also be performed. Referring to FIG. 4, FIG. 5, and FIGS. 6A to 6C, step S403′ may be performed by at least one of the medical imaging acquisition system (100), the data processing device (110′), the image projection system (150), or another computing device connected via wired or wireless communication means. Descriptions of technical steps overlapping with the embodiment of FIGS. 1 to 3 are omitted.

### <Step S404>

Next, a step (S404) of displaying the three-dimensional model on the augmented reality device (120) may be performed. As shown in FIG. 2 and FIG. 3, the three-dimensional model (301) is displayed in the first display area (210) of the augmented reality device (120), and the three-dimensional model (301) reflects the state of the patient lying in the prone posture.

In an embodiment based on a projection mapping method, a step (S404′) similar to step S404 may also be performed, although the technical configuration is somewhat different. Referring to FIG. 4, FIG. 5, and FIGS. 6A to 6C, in the embodiment based on the projection mapping method, a step (S404′) of directly projecting the three-dimensional model onto the body surface of the patient may be performed. The step (S404′) may utilize the body surface detection sensor (141), the gaze-tracking sensor (142), the projectors (151a, 151b), and the image registration and correction control unit (152) to project the three-dimensional model in real time in alignment with the patient's skin. In addition, even when the gaze of the user changes or the state of the body surface changes, the three-dimensional model may be updated in real time so that a natural stereoscopic effect is provided as if the user were actually viewing the interior of the body.

### <Step S405>

Next, a step (S405) of setting the display range of the three-dimensional model (301) may be performed. The display range setting step (S405) may be performed by (1) displaying, on the screen of the augmented reality device (120), a first list in which predetermined anatomical structures are enumerated and receiving a selection value input by the user, or (2) displaying the three-dimensional model (301) on the screen of the augmented reality device (120) and receiving a selection value input by the user, or (3) displaying, on the screen of the augmented reality device (120), a second list in which layers are enumerated and receiving a selection value input by the user.

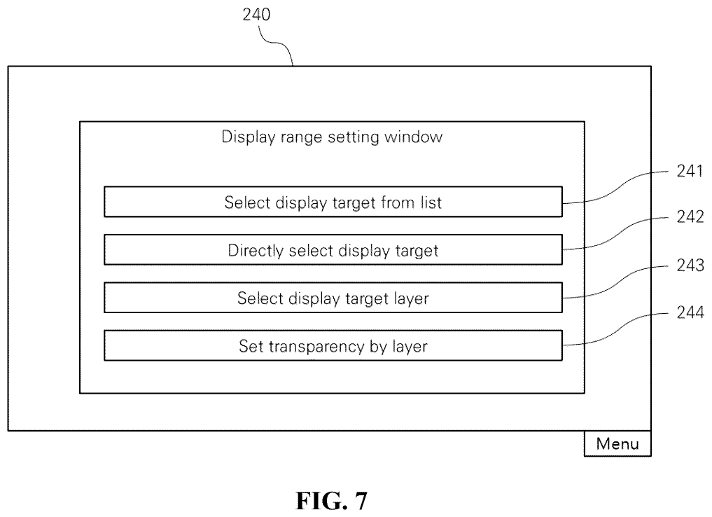

The display range setting step (S405) will be described with reference to FIG. 7. FIG. 7 is a diagram illustrating a fourth display area (240) (display range setting window). The display range setting of the three-dimensional model (301) relates to defining which portion of the three-dimensional model displayed on the augmented reality screen will be shown.

When a call to the “display range setting” menu is received from the user, the fourth display area (240) may be displayed on the augmented reality screen. On the selection screen, menus such as “select display target from list” (241), “directly select display target” (242), “select display target layer” (243), and “set transparency by layer” (244) may be enumerated.

When the “select display target from list” (241) menu is selected, a list of predetermined anatomical structures is displayed, and when a specific anatomical structure is selected from the list, only the portion of the three-dimensional model (301) corresponding to the selected anatomical structure may be displayed on the augmented reality screen. Alternatively, when the “directly select display target” (242) menu is selected, the three-dimensional model (301) is displayed in the fourth display area (240), and when the user selects a specific anatomical structure directly, only the portion of the three-dimensional model (301) corresponding to the selected anatomical structure may be displayed on the augmented reality screen.

For example, when the surgical target is around the fourth vertebra, if the third to fifth vertebrae are selected, only the portion of the three-dimensional model (301) corresponding to the third to fifth vertebrae may be displayed on the augmented reality screen. Accordingly, the user can perform surgery with greater focus on the surgical target. In addition, when a region of interest is selected, the anatomical structure at the boundary of the region of interest may be displayed in a cut-away form, allowing the anatomical structure within the region of interest to be reviewed from various angles.

Furthermore, when the “select display target layer” (243) menu is selected, only the layer of interest in the three-dimensional model (301) may be displayed in the fourth display area (240). The three-dimensional model (301) may include a plurality of layers, and the plurality of layers may include a skin layer of the patient, a bone layer, an intervertebral disc layer, a ligament layer, a nerve layer, and a vascular layer. When the “set transparency by layer” (244) menu is selected, the transparency of each layer may be set. For example, if the user wishes to view only the bone layer and the nerve layer, the other layers may be set to an off state or set to a highly transparent state.

In an embodiment based on a projection mapping method, a step (S405′) similar to step S405 (display range setting step) may also be performed. Referring to FIG. 4, FIG. 5, and FIGS. 6A to 6C, the display range setting step (S405′) may be performed by (1) displaying, on the screen of the display module (161), a first list in which predetermined anatomical structures are enumerated and receiving a selection value input by the user using the input interface module (162), or (2) displaying the three-dimensional model on the screen of the display module (161) and receiving a selection value input by the user using the input interface module (162), or (3) displaying, on the screen of the display module (161), a second list in which the layers are enumerated and receiving a selection value input by the user using the input interface module (162). Descriptions of technical steps overlapping with step S405 are omitted.

### <Step S406>

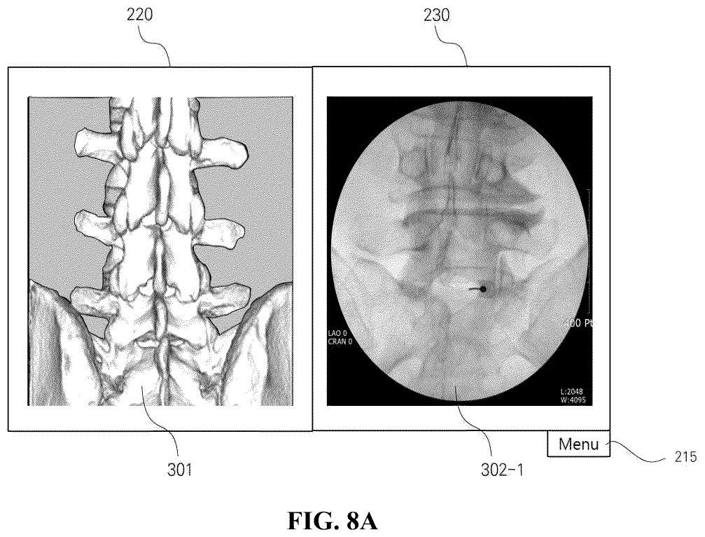

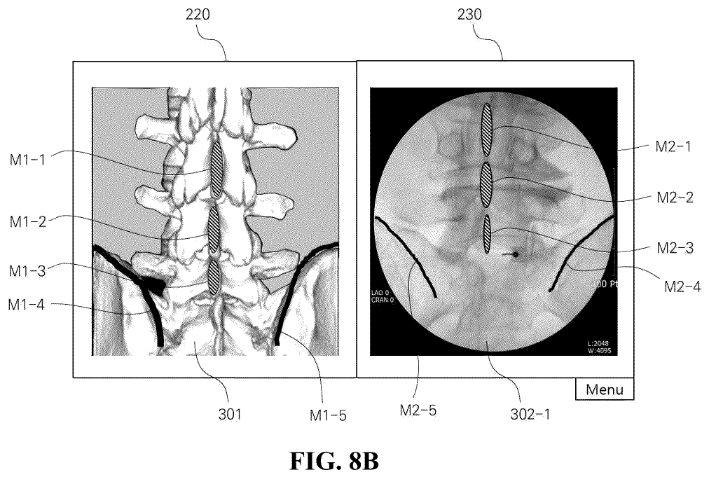

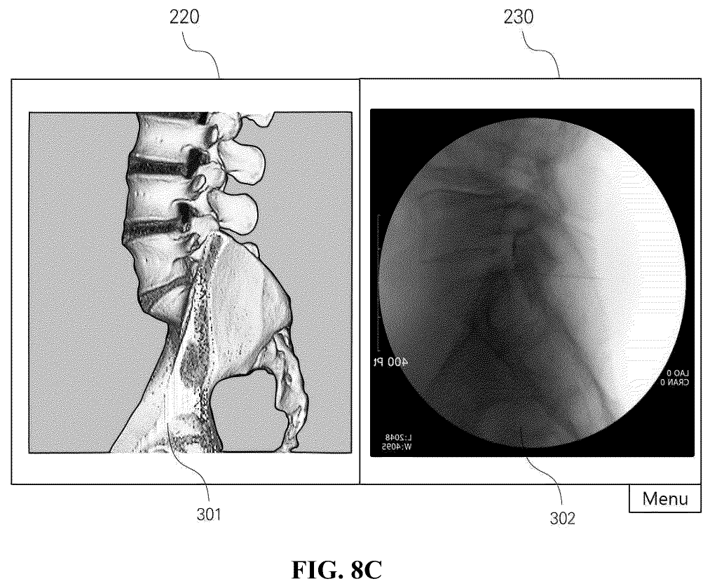

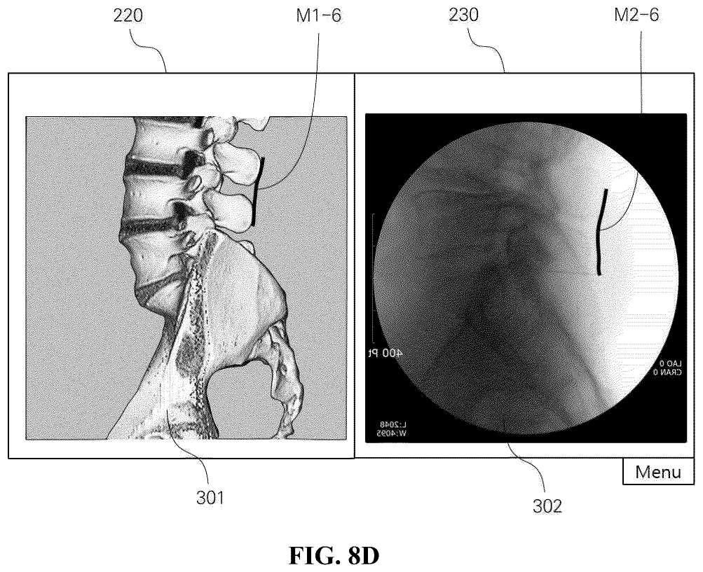

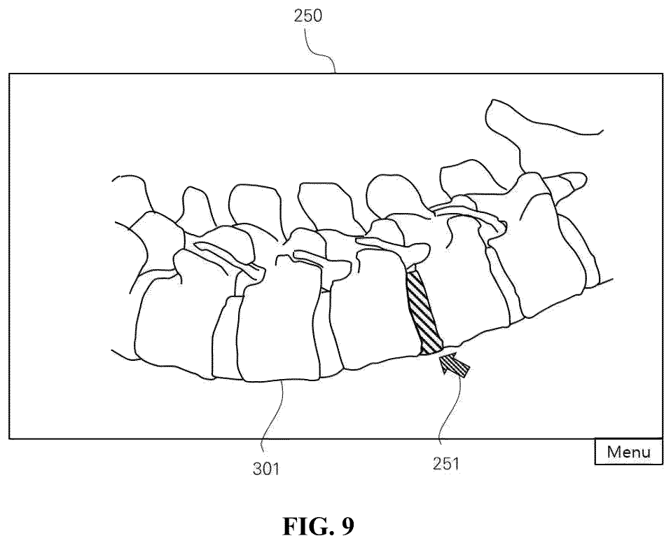

Steps S406, S407, S408, and S409 described below are steps performed in addition to the adjustment process in step S403, for the purpose of additional fine adjustment. Next, a step (S406) of receiving a first reference point with respect to the three-dimensional model may be performed. When it is desired to make the three-dimensional model more closely match the actual state of the patient, additional fine adjustment may be performed. This will be described with reference to FIGS. 8A to 8D.

FIGS. 8A to 8D are diagrams illustrating a process for setting reference points with respect to the three-dimensional model and the second image. FIG. 8A is an example of an image displayed in a reference point description window that appears when the user wearing the augmented reality device (120) activates the reference point setting mode.

As shown in FIG. 8A, the three-dimensional model (301) is displayed in the second display area (220) of the augmented reality device (120), and the user may input a first reference point with respect to a characteristic structure of the three-dimensional model (301). The term “characteristic structure” may refer to a tip of a spinous process or an iliac crest. Such anatomical structures are visually apparent and have distinctive shapes, and are therefore effective in the error correction process. The user may input the first reference point by marking a point or drawing a line with respect to the characteristic structure on the augmented reality screen.

As shown in FIG. 8B, a state is illustrated in which first reference points are marked with respect to characteristic structures such as the spinous process and the iliac crest. That is, the user may mark a plurality of first reference points (M1-1, M1-2, M1-3) for the spinous process and first reference points (M1-4, M1-5) for the iliac crest on the three-dimensional model (301) displayed in the second display area (220).

The input process by the user may be performed using hand gestures, voice, or a controller. For example, the user may point to the position of the reference point using fingers or hands, and the augmented reality device (120) may recognize the position and set it as a reference point. Alternatively, the process may be carried out using voice commands. For example, the reference point may be set using a voice command such as “set this point as a reference point.” The process may also be performed by using a controller (not shown) of the augmented reality device (120) to move a pointer on the screen to the position of the reference point and pressing a setting button.

In another embodiment, the step of setting the first reference point may be performed by allowing the user to select from a predetermined list of anatomical structures. When a selection window displaying a list of multiple anatomical structures is presented, the user may select the desired anatomical structure through hand gestures, voice, or other means. If the user selects “spinous process,” an additional selection window may be displayed to allow the user to select which spinous process is intended. For example, in the case of the cervical vertebrae, a list of C1 to C7 may be displayed; in the case of the thoracic vertebrae, a list of T1 to T12 may be displayed; in the case of the lumbar vertebrae, a list of L1 to L5 may be displayed; and in the case of the sacral vertebrae, a list of S1 to S5 may be displayed. When the user selects a specific spinous process, a marking may be displayed on the three-dimensional model. For example, if the user selects L4 and L5 of the lumbar vertebrae, markings may be automatically displayed at the ends of the lumbar vertebrae L4 and L5 on the three-dimensional model. Subsequently, if the automatically displayed positions of the markings on the three-dimensional model are inaccurate, a step of finely adjusting the markings may be performed through hand gestures, voice commands, or controller manipulation.

In another embodiment, instead of opening a window for selecting an anatomical structure, the user may directly set a marking at a specific position on the three-dimensional model (301). That is, a method of marking a point or a line on a specific portion of the three-dimensional model (301) displayed in the second display area (220) may be applied.

In an embodiment based on a projection mapping method, a step (S406′) similar to step S406 may also be performed. FIGS. 8A and 8B may be examples of images displayed in a reference point description window that appears when a reference point setting mode is activated on the display module (161). The input process by the user may be performed using the input interface module (162). Descriptions of technical steps overlapping with step S406 are omitted.

### <Step S407>

Next, a second reference point reception step (S407) may be performed, in which the second image data is visually displayed on the screen of the augmented reality device (120), and second reference points input by the user with respect to characteristic structures of the second image data are received.

This will be described with reference to FIGS. 8A to 8D. The second reference point reception step (S407) may be further divided as follows:
(1) visually displaying, on the screen of the augmented reality device (120) (third display area (230)), second-1 image data (302-1) photographed in a direction viewing the patient from above downward (see the third display area (230) of FIG. 8A), and receiving second-1 reference points (M2-1, M2-2, M2-3, M2-4, M2-5) input by the user with respect to characteristic structures of the second-1 image data (302-1); (2) visually displaying, on the screen of the augmented reality device (120) (third display area (230)), second-2 image data (302-2) photographed in a direction viewing the flank of the patient; and
(3) receiving a second-2 reference point (M2-6) input by the user with respect to characteristic structures of the second-2 image data (302-2).

FIG. 8D illustrates an example in which reference points are input in the form of a curve formed by a plurality of spinous processes. When a plurality of spinous processes are input as reference points, the system may be configured to automatically generate a curve formed by the spinous processes. In addition, when a curve is input, the ends of the plurality of spinous processes may be set to be positioned on the input curve.

In an embodiment based on a projection mapping method, a step (S407′) similar to step S407 may also be performed. In the second reference point reception step (S407′), the second image data is visually displayed on the screen of the display module (161), and second reference points input by the user with respect to characteristic structures of the second image data are received through the input interface module (162).

### <Step S408>

Next, a fine adjustment step (S408) of adjusting the three-dimensional model (301) using the first reference points and the second reference points may be performed. The fine adjustment step (S408) may include a step (S408-1) of mapping the correspondence between the first reference points (M1-1, M1-2, M1-3, M1-4, M1-5, M1-6) and the second reference points (M2-1, M2-2, M2-3, M2-4, M2-5, M2-6), a step (S408-2) of estimating the second posture based on the mapped correspondence, and a step (S408-3) of adjusting the three-dimensional model (301) according to the estimated second posture.

Descriptions of the specific algorithms for performing steps S408-1, S408-2, and S408-3 are already included in the explanation of the three-dimensional model adjustment step (S403), and thus redundant descriptions are omitted.

In an embodiment based on a projection mapping method, a step (S408′) similar to step S408 may also be performed. The step (S408′) may be performed by at least one of the data processing device (110′), the image registration and correction control unit (152), the interface controller (163), or another computing device connected via wired or wireless communication means. Descriptions of technical steps overlapping with step S408 are omitted.

### <Step S409>

Next, an additional adjustment step (S409) during surgery may be performed. This step may be carried out when the posture of the patient changes during surgery or when reconfirmation of position is required, and the adjustment process may be performed in the same manner as step S403 or in the same manner as steps S406, S407, and S408.

In an embodiment based on a projection mapping method, a step (S409′) similar to step S409 may also be performed. When the posture of the patient changes during surgery, the step (S409′) may be carried out when reconfirmation of position is required, and the adjustment process may be performed in the same manner as step S403′ or in the same manner as steps S406′, S407′, and S408′. In addition, the additional adjustment step may also be performed by utilizing the body surface detection sensor (141) and the image registration and correction control unit (152).

### <Step S410>

Next, a surgical target setting step (S410) of setting a surgical target with respect to the three-dimensional model may be performed. This step may be carried out in advance before the start of surgery. For example, in the case of applying endoscopic discectomy surgery, the disc or bone (lamina) compressing the nerve is the surgical target, and this step may correspond to designating the surgical target on the three-dimensional model. If there is a portion of the disc that has deformed and is compressing the surrounding nerve, the portion compressing the nerve may be input as the region to be removed (surgical target).

When the user selects the surgical target setting menu through the augmented reality device (120), the surgical target setting menu may be invoked and displayed on the screen of the augmented reality device (120) (for example, in the fifth display area (250)). The surgical target setting menu may relate to anatomical structures. In order for this step to be performed, a step of automatically classifying the three-dimensional model according to anatomical structures needs to be executed in advance. When the user selects a desired item from the invoked surgical target setting menu, the selected item may be stored as a surgical target model.

In another embodiment, the step may include storing as a surgical target model a region designated by the user with respect to the three-dimensional model (301) displayed on the augmented reality device (120). More specifically, when the user designates a region in various ways on the virtual screen, a portion of the three-dimensional model included in the designated region may be stored as the surgical target model. Methods of designating the region may include a method of clicking a specific position on the screen and a method of selecting a specific area on the screen. The user's input method may be a hand gesture, a voice command, or the use of a controller.

In FIG. 7, the three-dimensional model (301) is displayed in the fifth display area (250) (surgical target setting window), and a cursor (251) movable by the user is displayed. When the user moves the cursor (251) and clicks on an intervertebral disc, the entire intervertebral disc may be selected as the surgical target model. The fifth display area (250) may be the same as the first display area.

In an embodiment based on a projection mapping method, a step (S410′) similar to step S410 may also be performed. In the step (S410′), when the user selects the surgical target setting menu through the input interface module (162), the surgical target setting menu may be invoked and displayed on the screen of the display module (161).

### <Step S411>

Next, an insertion path computation step (S411) of computing an optimal surgical instrument insertion path toward the surgical target may be performed. The insertion path computation step (S411) may include a step (S411-1) of dividing the three-dimensional model (301) into a plurality of small voxels, a step (S411-2) of storing an initial position from which the surgical instrument (SI) is inserted and a surgical target position to which the surgical instrument (SI) is to reach, a cost assignment step (S411-3) of assigning movement costs to each voxel, and a step (S411-4) of computing costs required to pass through voxels between the initial position and the surgical target position and determining, as the optimal surgical instrument insertion path, the path with the lowest cost.

The cost assignment step (S411-3) may be performed in such a way that relatively high costs are assigned to voxels adjacent to predetermined anatomical structures. In addition, voxels located at positions into which the surgical instrument (SI) cannot be inserted or is difficult to insert may also be assigned relatively high costs.

The insertion path computation step (S411) will now be described in more detail. Selecting the optimal surgical instrument insertion path is important in minimally invasive spine surgery. Minimally invasive spine surgery is a field of spinal surgery in which surgery is performed through smaller incisions compared to traditional open surgery, thereby shortening the patient's recovery time and minimizing postoperative pain and complications.

Embodiments of the present invention may be applied to minimally invasive spine surgery techniques such as microdiscectomy, percutaneous pedicle screw fixation, endoscopic discectomy, percutaneous laser disc decompression (PLDD), vertebroplasty, kyphoplasty, and artificial disc replacement. In embodiments of the present invention, cases of applying endoscopic discectomy are mainly described.

The surgical instrument (SI) refers to tools used in each surgical technique. For example, in the case of applying endoscopic discectomy, the surgical instrument (SI) may be a needle, a dilator, an endoscope, forceps, a laser probe, or a radiofrequency probe. The surgical instrument insertion path (261) refers to a path along which the surgical instrument (SI) can be safely inserted toward the surgical target. Factors reflected in the computation process of the surgical instrument insertion path (261) may include the positions of major neural tissues, major vascular tissues, and bone tissues, as well as the minimum insertion distance required for minimally invasive surgery. It is important to prevent major nerves or organs from being damaged when the surgical instrument (SI) is inserted from the flank or the back. In the case of endoscopic discectomy, it is important to derive the optimal surgical instrument insertion path (optimal position and angle) by considering the shape and position of the disc that is the surgical target, the shapes and positions of the surrounding bone tissues and neural tissues, and the shapes and positions of the surrounding vascular tissues.

As path-finding algorithms that may be used in the insertion path computation step (S411), algorithms such as the A* algorithm, the Dijkstra algorithm, the RRT (Rapidly-exploring Random Tree) algorithm, or a Genetic Algorithm may be applied. Hereinafter, the A* algorithm will be described as an example.

First, a step (S411-1) of dividing the three-dimensional model into small voxels may be performed. The size of the voxels may be appropriately determined according to the precision required in path computation. Then, a process (S411-3) of assigning movement costs to each voxel may be performed. For example, higher costs may be assigned to regions near critical nerves or blood vessels, so that such regions are avoided during path searching. In addition, regions to be avoided (such as bones or blood vessels) may be input as obstacles or may be assigned high costs.

Further, an initial position where the surgical instrument (SI) is inserted through the skin and a surgical target position where the surgical instrument (SI) is to reach may be input (S411-2). If there are physical constraints such as restrictions that the surgical instrument (SI) cannot bend beyond a certain angle, a step of reflecting such constraints may be performed. A step of executing a function for calculating estimated costs from the initial position to the surgical target may be performed, and costs for multiple paths may be computed so that the path with the lowest cost is determined as the optimal path (S411-4).

In an embodiment based on a projection mapping method, a step (S411′) similar to step S411 may also be performed. The step (S411′) may be performed by at least one of the data processing device (110′) or another computing device connected via wired or wireless communication means. Descriptions of technical configurations overlapping with step S411 are omitted.

### <Step S412>

Next, an insertion path display step (S412) of displaying the optimal surgical instrument insertion path (261) on the screen of the augmented reality device (120) may be performed. In particular, on the screen of the augmented reality device (120), the optimal surgical instrument insertion path (261) may be displayed overlapped with the actual appearance of the patient. This step corresponds to displaying the optimal surgical instrument insertion path (261) on the three-dimensional model (301). When the user selects the “surgical instrument insertion path” menu, a sixth display area (260) (surgical instrument insertion path window) is opened, and the optimal surgical instrument insertion path (261) may be displayed in the sixth display area (260). In another embodiment, the optimal surgical instrument insertion path (261) may be displayed overlapped with the actual appearance of the patient in the first display area (210) (main window).

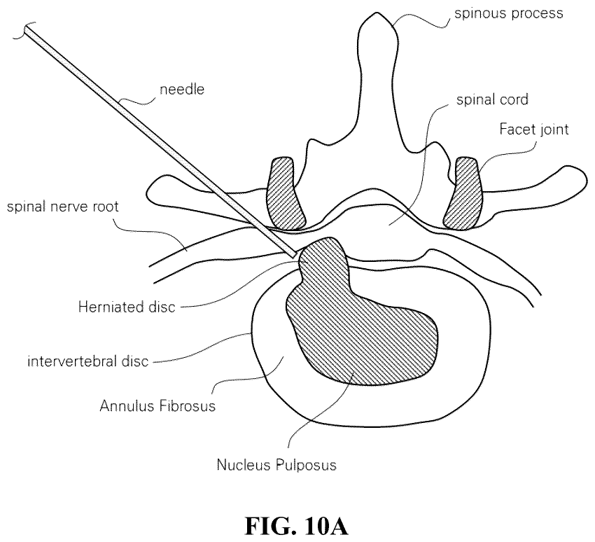

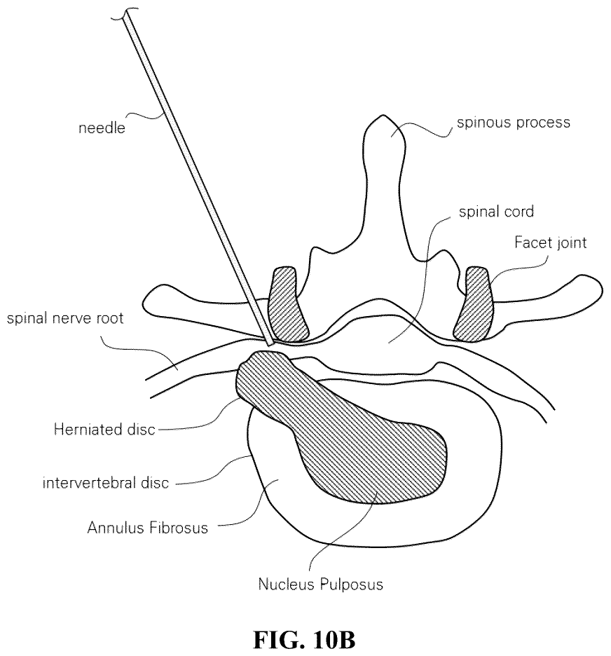

The insertion path display step (S412) will be described in detail with reference to FIGS. 10A and 10B. FIG. 10A shows a state in which a herniated disc is located on the upper surface of an intervertebral disc. In such a state, since a facet joint of the spine is located on the path along which the surgical instrument (for example, a needle) approaches the herniated disc, it is difficult to insert the surgical instrument. In order to avoid the facet joint and insert the surgical instrument to reach the herniated disc, the surgical instrument needs to be inserted at an angle of about 20 to 45 degrees.

FIG. 10B shows a state in which a herniated disc is located on the lateral surface of an intervertebral disc. In such a case, even if the surgical instrument (for example, a needle) is inserted at an angle close to vertical, it is easy to avoid the facet joint and insert the surgical instrument to reach the herniated disc.

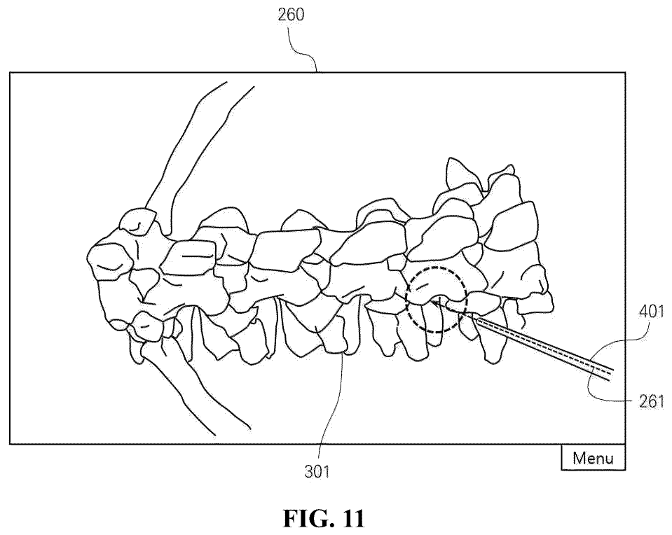

FIG. 11 is a diagram for explaining the optimal surgical instrument insertion path displayed in the sixth display area (260). In FIG. 11, the three-dimensional model (301) and the optimal surgical instrument insertion path (261) are displayed together in the sixth display area (260) (surgical instrument insertion path window). The optimal surgical instrument insertion path (261) may be represented as a straight line or a curve in the sixth display area (260). The user may insert the surgical instrument into the human body by using such a straight line or curve as a guide. The sixth display area (260), in which the optimal surgical instrument insertion path (261) is displayed, may be the same area as the first display area (210).

The user may rotate the displayed three-dimensional model in multiple directions to identify the precise insertion path. The user may also zoom in or zoom out the displayed three-dimensional model to examine the detailed insertion path.

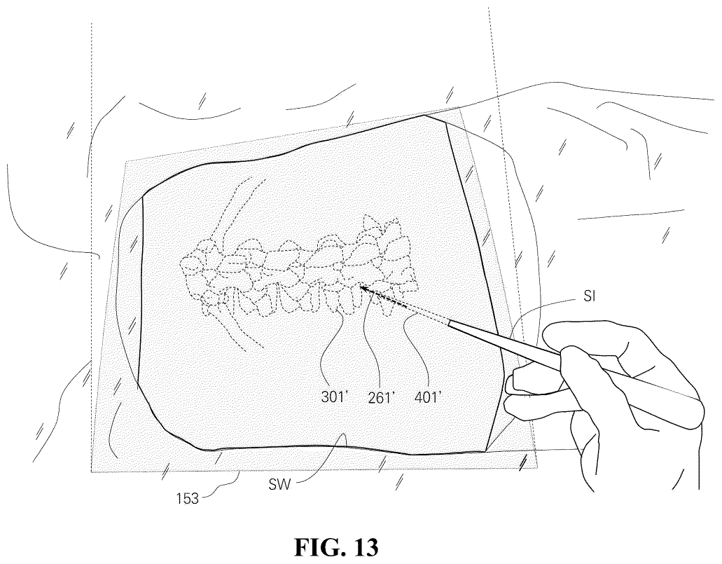

In an embodiment based on a projection mapping method, a step (S412′) similar to step S412 may also be performed. FIG. 13 is a diagram for explaining a projection image area and an optimal surgical instrument insertion path in an embodiment of the projection mapping method. Referring to FIG. 13, in the step (S412′), an insertion path display step of displaying the three-dimensional model (301′) and the optimal surgical instrument insertion path (261′) in the projection image area (153) may be performed. That is, the optimal surgical instrument insertion path (261′) may be displayed directly on the body surface of the patient. It is preferable to determine the number and installation angles of the projectors so that shadows of the surgical instrument (SI) are not formed on the optimal surgical instrument insertion path (261′) in the projection image area (153). In another embodiment, the three-dimensional model and the optimal surgical instrument insertion path may be displayed on the screen of the display module (161). The user may rotate, zoom in, or zoom out the three-dimensional model displayed on the screen of the display module (161) in order to identify the accurate insertion path. Descriptions of technical configurations overlapping with step S412 are omitted.

### <Step S413>

Next, a surgical instrument model display step (S413) of displaying a surgical instrument model (401), which represents the surgical instrument (SI) in three dimensions, on the screen of the augmented reality device (120) may be performed. The surgical instrument model display step may include a step (S413-1) of detecting the surgical instrument from the second image data and a step (S413-2) of displaying the detected surgical instrument as a surgical instrument model overlapped with the three-dimensional model. On the screen of the augmented reality device (120), the optimal surgical instrument insertion path (261) and the surgical instrument model (401) may be displayed overlapped together.

The surgical instrument model (401) is a three-dimensional representation of the actual surgical instrument inserted into the human body. To generate the surgical instrument model, three-dimensional scan data of the surgical instrument to be used may be pre-input. Alternatively, when the surgical instrument is inserted into the human body, the shape of the surgical instrument may be detected, the most similar surgical instrument may be identified, and the corresponding surgical instrument model may be displayed. More specifically, when the surgical instrument is inserted into the human body, X-ray image data (the second image data) may be acquired using the C-arm, and accordingly, the shape of the inserted surgical instrument may be detected. Thus, the position and angle of the surgical instrument may be determined, and the surgical instrument model (401) may be displayed overlapped with the three-dimensional model (301) as a background. In another embodiment, a position-tracking sensor for detecting the position and angle of the surgical instrument may be used to identify the real-time position and angle of the surgical instrument and update the state of the surgical instrument model (401).

In general, it is difficult for the user to recognize the position of a surgical instrument inserted into the human body during surgery. However, when the optimal surgical instrument insertion path (261) and the surgical instrument model (401) are simultaneously displayed (overlapped) in the same display area, safer and more efficient surgery becomes possible. Conventionally, C-arm images are presented separately as planar images, such as a top-down image (viewing the back of the patient from above) and a lateral image (viewing the flank of the patient from the side). Accordingly, it has been difficult for the user to intuitively determine the three-dimensional position and angle of the inserted surgical instrument.

However, according to an embodiment of the present invention, the user can easily and intuitively determine the current three-dimensional position of the surgical instrument. By viewing the surgical instrument model (401) together with the three-dimensional model (301), the user can guide the surgical instrument accurately and safely toward the surgical target.

The display process of the surgical instrument will be described in more detail. The initial insertion point of the surgical instrument (SI) may be detected through C-arm image acquisition, and when the surgical instrument is inserted, C-arm image acquisition may be performed from multiple angles. Based on multi-angle C-arm images, a three-dimensional model of the surgical instrument (surgical instrument model) may be generated, and the position and angle of the surgical instrument may be derived. Then, the position and shape of the surgical instrument model may be updated in real time, and the surgical instrument model may be merged with the three-dimensional model of the patient and displayed. Three-dimensional visualization tools such as OpenGL, VTK, or Unity may be used to render the model.

In another embodiment, a position-tracking sensor for detecting the position and angle of the surgical instrument may be used to identify the real-time position and angle of the surgical instrument and update the state of the surgical instrument model (401).

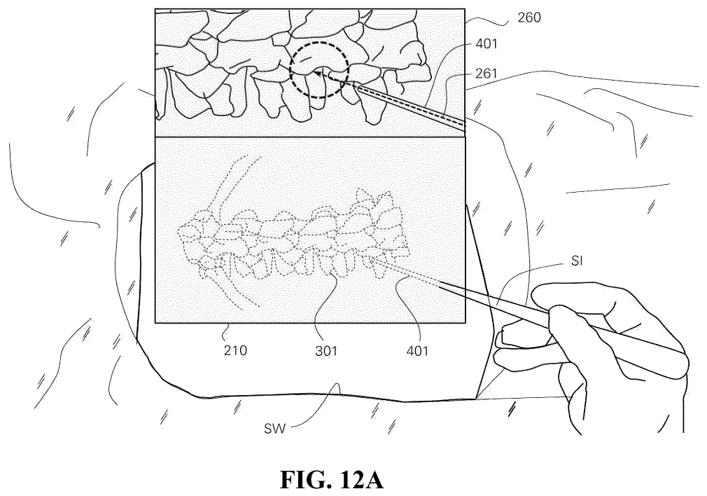

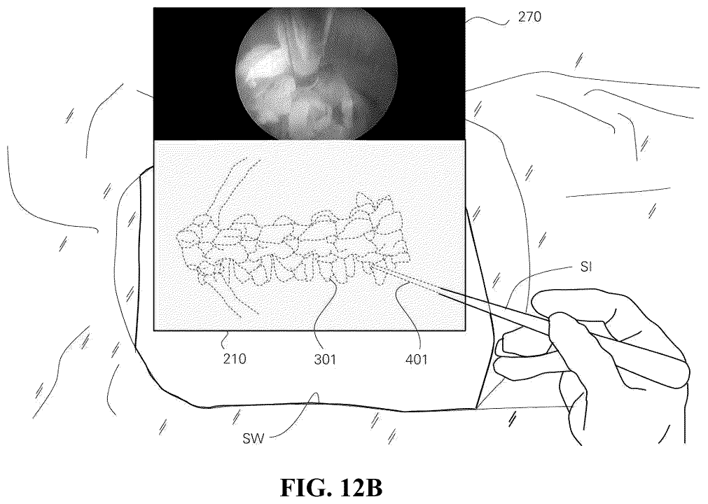

As shown in FIG. 12A, the three-dimensional model (301) and the surgical instrument model (401) are displayed in the first display area (210), and the actual appearance of the patient may be overlapped together. In addition, in the sixth display area (260) located at the upper portion of the augmented reality screen, the three-dimensional model (301), the surgical instrument model (401), and the optimal surgical instrument insertion path (261) may be displayed as a more enlarged image or as an image viewed from another angle. Accordingly, the user can more easily determine from which direction and at what angle the surgical instrument should be inserted to be safe and effective.

In an embodiment based on a projection mapping method, a step (S413′) similar to step S413 may also be performed. Referring to FIG. 13, the step (S413′) (surgical instrument model display step) is a step of displaying a surgical instrument model (401′), which represents the surgical instrument (SI) in three dimensions, in the projection image area (153). The surgical instrument model display step (S413′) may include a step (S413-1′) of receiving surgical instrument information and a step (S413-2′) of forming the surgical instrument model (401′) based on the received surgical instrument information and displaying it overlapped with the three-dimensional model. In the projection image area (153), the optimal surgical instrument insertion path (261′) and the surgical instrument model (401′) may be displayed overlapped together. By using the position-tracking sensor (143) to identify the position and angle of the surgical instrument, the surgical instrument model (401′) may be displayed overlapped with the three-dimensional model (301′) as a background. In another embodiment, X-ray image data (the second image data) may be acquired using the C-arm, and the position and angle of the inserted surgical instrument may be identified, and accordingly, the surgical instrument model (401′) may be displayed overlapped with the three-dimensional model (301′) as a background. Descriptions of technical configurations overlapping with step S413 are omitted.

### <Step S414>

Next, a step (S414) of visually displaying third image data acquired by the third medical imaging device (105) on the screen of the augmented reality device (120) may be performed. The third medical imaging device (105) may be an endoscopic camera.

As shown in FIG. 12B, real-time endoscopic camera images may be displayed in the seventh display area (270) of the augmented reality device (120). The user may perform surgery while simultaneously checking the first display area (210) and the seventh display area (270). Accordingly, since the user can simultaneously check the three-dimensional model (301), the surgical instrument model (401), the endoscopic camera images, and the actual appearance of the patient, it is easy to determine the exact state (position, angle, etc.) of the surgical instrument. Of course, depending on the embodiment, the seventh display area (270) may be the same as the first display area (210).

In an embodiment based on a projection mapping method, a step (S414′) similar to step S414 may also be performed. The step (S414′) is a step of visually displaying the third image data acquired by the third medical imaging device (105) on the interface system, particularly on the display module (161). The third medical imaging device (105) may be an endoscopic camera. Descriptions of technical configurations overlapping with step S414 are omitted.

### <Step S415>

Next, a surgical state reflection step (S415) of displaying a three-dimensional model (301) reflecting the surgical state on the screen (eighth display area (280)) of the augmented reality device may be performed.

The surgical state reflection step (S415) may be a step of computing surgical progress using at least one of the number of times the surgical instrument model (401) passes through each position, the three-dimensional range in which the surgical instrument model (401) has moved, and the residence time of the surgical instrument model (401) at each position, and reflecting on the surgical target model the region in which surgery has been performed according to the surgical progress. In addition, depending on the surgical progress, the color of the surgical target model may be displayed differently, or the transparency of the surgical target model may be displayed differently.

Furthermore, when the surgical instrument model (401) approaches within a predetermined distance of a predetermined anatomical structure, or when the surgical progress reaches a preset level, a step of providing a notification to the user through the augmented reality device (120) may be performed.

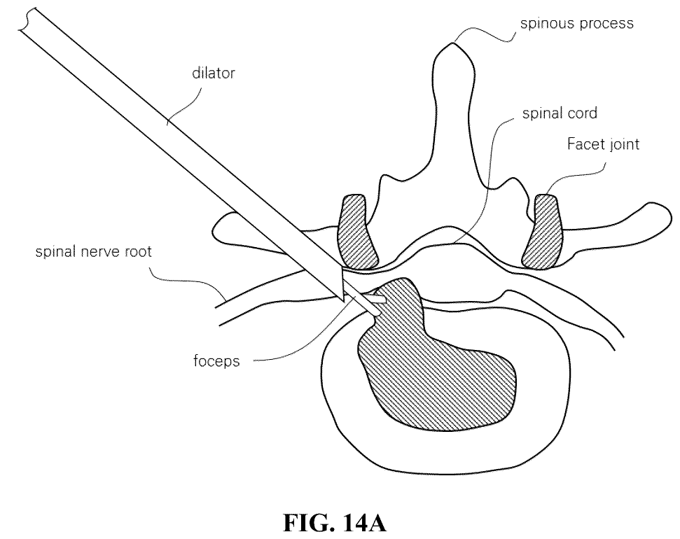

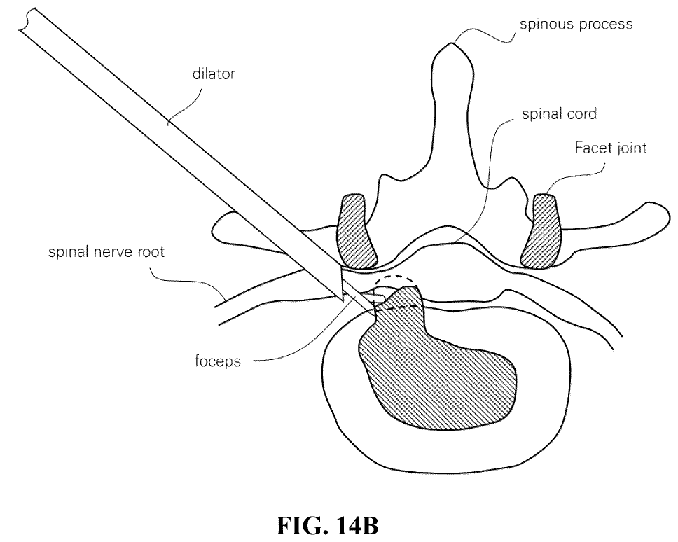

With reference to FIGS. 14A, 14B, 15, 16, 17A, and 17B, the details will now be described. FIGS. 14A and 14B are diagrams illustrating the procedure of endoscopic discectomy. The procedure of endoscopic discectomy is as follows. The user inserts a needle into the spinal disc while exploring the path. During this process, fluoroscopy (C-arm) may be used to confirm the position of the needle in real time. When the needle reaches the correct position, a trocar is inserted along the path. Once the trocar is placed in the correct position, the needle is removed. Through the trocar, a tubular dilator is used to gradually expand the path. When the path is sufficiently secured, the user inserts an endoscope. While viewing the third image (endoscopic camera image), the user removes the herniated disc that is compressing the spinal nerve root. Specifically, the user inserts surgical instruments such as forceps, cutters, lasers, or radiofrequency probes through the inside of the dilator and removes the protruding herniated disc. Accordingly, the compression on the spinal nerve root is relieved, thereby reducing pain.

The user needs to accurately identify the state of the surgical target (herniated disc) during the surgical procedure. Currently, the removal of the disc is performed relying on the intuition of the user. Since the endoscope provides only an enlarged view of a partial region, it is difficult to precisely determine which portion has been removed. As a result, excessive removal of the disc (or nucleus pulposus) may occur, or conversely, the disc may not be sufficiently removed. According to an embodiment of the present invention, since the user can visually and clearly identify the removed range of the disc and the remaining range of the disc, more accurate surgery may be performed.

The process of reflecting the surgical progress on the three-dimensional model may proceed as follows. First, the three-dimensional model (301) is prepared. Next, the position of the surgical instrument (SI) is tracked and confirmed through real-time C-arm images (second image data). The three-dimensional surgical instrument model (401) is integrated into the three-dimensional model (301) of the patient. The patient's three-dimensional model (301) and the surgical instrument model (401) are registered so that the exact position and orientation of the surgical instrument (SI) are mapped. Next, the three-dimensional model is rendered using three-dimensional visualization libraries such as VTK, Unity, or Three.js. Next, based on the movement and interaction of the surgical instrument model (401), the region in which surgery has been performed (for example, the region of herniated disc tissue that has been removed) is detected.

For surgical targets that cannot be detected through C-arm images (for example, the herniated disc itself), surgical progress may be computed by comprehensively scoring data such as the three-dimensional region traversed by the surgical instrument model, the number of times the surgical instrument model has passed through, and the working time of the surgical instrument model. Next, the initial state of the surgical target (for example, the initial volume of the intervertebral disc) and the current state of the surgical target (for example, the current volume of the intervertebral disc) may be compared to calculate the amount of change. Next, the region where surgery has been performed (for example, the removed herniated disc region) may be displayed in a different color. For example, the original nucleus pulposus (or intervertebral disc) may be displayed in green, and the removed nucleus pulposus region may be displayed in red. Alternatively, the transparency of the region where surgery has been performed (for example, the removed herniated disc region) may be varied to clearly visualize the relationship with other anatomical structures. In addition, during the surgical process (for example, the removal of the herniated disc), the state of change of the surgical target (for example, the herniated disc) may be displayed step by step according to the surgical progress. For example, the color may gradually change or the transparency may gradually change. Furthermore, the amount of the removed herniated disc (or disc) may be quantitatively calculated and provided to the user. A function of providing visual or auditory warnings may also be added when approaching important nerves or blood vessels. Furthermore, when the amount of removed herniated disc reaches a predetermined target, a notification may be provided.

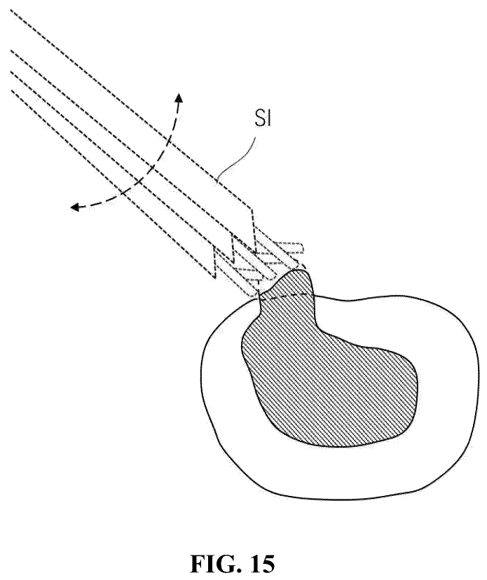

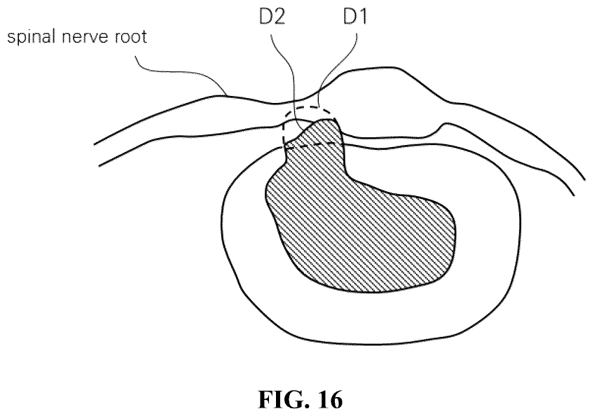

FIG. 15 illustrates the procedure of endoscopic discectomy. A herniated disc is removed by inserting forceps through the dilator. The herniated disc compresses the spinal nerve root, and as the herniated disc is removed, the pressure applied to the spinal nerve root is relieved. FIG. 16 shows the state in which the herniated disc is reduced as the herniated disc is removed using forceps. The region in which surgery has been performed (for example, the region where disc tissue has been removed) may be estimated based on the movement and interaction of the surgical instrument model. Referring to FIG. 15, the three-dimensional range through which the surgical instrument model has moved is detected, and accordingly, the range of the removed herniated disc may be derived. By comprehensively scoring data such as the three-dimensional region traversed by the surgical instrument model, the number of times the surgical instrument model has passed through, and the working time of the surgical instrument model, the work progress with respect to the surgical target may be computed.

As shown in FIG. 16, D1 represents the initial state of the surgical target (the initial three-dimensional shape of the intervertebral disc, which is the surgical target), and D2 represents the current state of the surgical target (the current three-dimensional shape of the intervertebral disc, which is the surgical target). The initial state of the surgical target is the initial shape of the intervertebral disc (disc, surgical target) identified from the three-dimensional model before the start of surgery. The current state of the surgical target may be estimated as the region where surgery has been performed (for example, the region where the herniated disc has been removed) based on the movement and interaction of the surgical instrument model. That is, the region into which the surgical instrument has entered, moved sufficiently, and performed operations may be estimated as the region in which disc tissue has been removed. The residence time of the surgical instrument at each position and the number of passes of the surgical instrument may be reflected as evaluation factors and scored. A high score may be estimated as indicating a high probability that the protruding intervertebral disc tissue has been removed. If the calculated score for a region is greater than or equal to a set threshold, the protruding intervertebral disc tissue in that region may be displayed as having been removed. A region into which the surgical instrument has not sufficiently entered will be calculated as having a score less than or equal to the threshold, and such a region may be displayed as a region in which the disc tissue has not been removed. Referring to FIG. 16, the three-dimensional range through which the surgical instrument model has moved, the residence time of the surgical instrument model, and the number of passes of the surgical instrument model at each position are detected, and accordingly, the range of the removed nucleus pulposus may be derived.

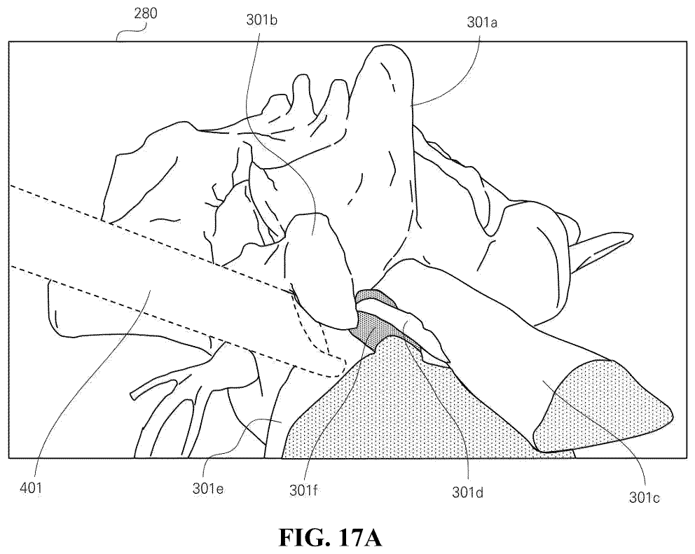

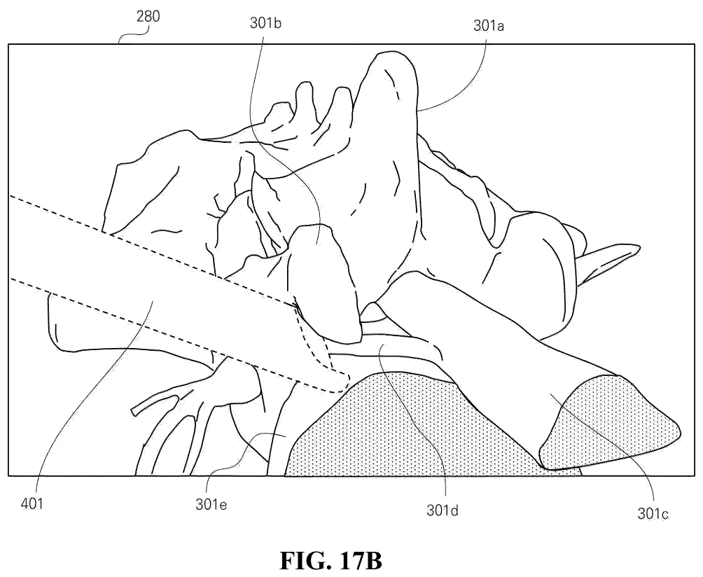

FIGS. 17A and 17B are diagrams illustrating a state in which the condition of the surgical target is reflected in three dimensions in real time and displayed in the eighth display area.

As shown in FIGS. 17A and 17B, the eighth display area (280) is an area displayed on the augmented reality device (120) and is a window for confirming the current state of the surgical target. The user may perform surgery with reference to the three-dimensional shape of the surgical target displayed in the eighth display area (280). FIG. 17A shows the state of the eighth display area (280) at the beginning of surgery. In the intervertebral disc (301e) (surgical target) of the three-dimensional model (301), the herniated disc (301f) is displayed. The herniated disc (301f) compresses the spinal nerve root (301d), thereby causing pain.

Referring to FIGS. 17A and 17B, the state of the surgical target may be reflected in three dimensions in real time in the eighth display area (280) according to the surgical progress. FIG. 17B shows a state in which the herniated disc (301f) has been completely removed, represented as a three-dimensional model. Accordingly, it shows a state in which the compression applied to the spinal nerve root (301d) has been released. The user may check the state of the surgical target displayed in the eighth display area (280) to confirm the progress of the surgery. As described above, the region in which surgery has been performed (for example, the region where the herniated disc has been removed) may be estimated based on the movement and interaction of the surgical instrument model. That is, the region into which the surgical instrument has entered, moved sufficiently, and performed operations may be estimated as the region in which disc tissue has been removed. The three-dimensional range through which the surgical instrument model has moved, the residence time of the surgical instrument model, and the number of passes of the surgical instrument model at each position are detected, and accordingly, the range of the removed nucleus pulposus may be derived.

In an embodiment based on a projection mapping method, a step (S415′) similar to step S415 may also be performed. The differences from step S415 will be described. The step (S415′) is a step of displaying a three-dimensional model (301′) reflecting the surgical state on the screen of the interface system (160), particularly on the display module (161). When the surgical instrument model (401′) approaches within a predetermined distance of a predetermined anatomical structure, or when the surgical progress reaches a preset level, a step of providing a notification to the user through the display module (161) may be performed. By using the position-tracking sensor (143), the real-time position of the surgical instrument (SI) may be tracked and confirmed, and the three-dimensional surgical instrument model (401′) may be integrated into the three-dimensional model (301′) of the patient. In FIGS. 17A and 17B, the eighth display area (280) may be an area displayed on the interface system (160), specifically the display module (161), in this embodiment.

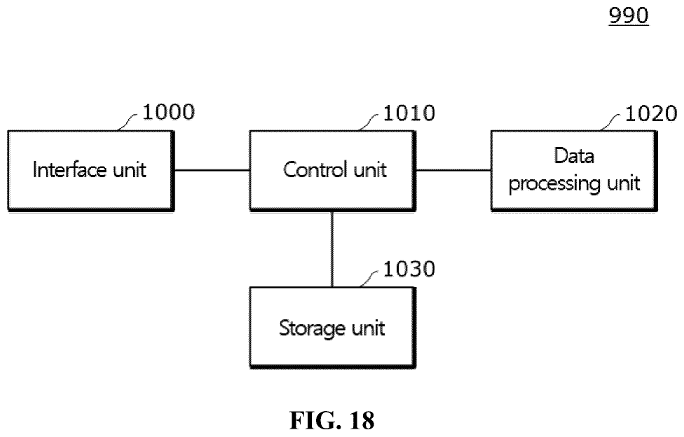

FIG. 18 is a block diagram illustrating a detailed structure of a spinal surgery image-providing system according to an embodiment of the present invention.

As shown in FIG. 18, the spinal surgery image-providing apparatus (990) according to an embodiment of the present invention is at least one device among the plurality of medical imaging devices (101, 103, 105), the data processing device (110), the augmented reality device (120), and the interface system (130) of FIG. 1, and includes some or all of an interface unit (1000), a control unit (1010), a data processing unit (1020), and a storage unit (1030).

Here, the phrase “includes some or all” means that the spinal surgery image-providing apparatus (990) may be configured by omitting certain components such as the storage unit (1030), or that certain components such as the data processing device (110) may be integrated into other components such as the control unit (1010). For sufficient understanding of the invention, however, the description is made with all components included.

For convenience of explanation, the spinal surgery image-providing apparatus (990) will be described below on the assumption that it is the data processing device (110) or the augmented reality device (120) of FIG. 1. For example, in the case of the augmented reality device (120), it is possible that the data processing device (110) is mounted inside. Thus, embodiments of the present invention are not particularly limited to any one form. However, in the case of the augmented reality device (120), it is preferable that the data be processed by the data processing device (110) and by user interface operations.

For example, the interface unit (1000) may include a communication interface for communicating with the medical imaging devices (101, 103, 105) of FIG. 1 and a display unit of the augmented reality device (120) for displaying images according to an embodiment of the present invention. In embodiments of the present invention, however, it is preferable that the data processing device (110) displays on the screen various setting windows described earlier through the user interface with the augmented reality device (120), and operates to allow setting of reference points and the like.

The interface unit (1000) of the data processing device (110) may receive data related to the three-dimensional model acquired by photographing the internal structure of the patient using the first medical imaging device (101) and provide the data to the control unit (1010). The interface unit (1000) may also receive image data of a two-dimensional image photographed by the second medical imaging device (103) and provide it to the control unit (1010). Furthermore, endoscopic images received from the third medical imaging device (105) may be provided to the control unit (1010).

The control unit (1010) may include processors such as a CPU, an MPU, or a GPU, and may further include memory such as RAM. Here, the processor and the memory may be integrated into a single chip in the form of an IC chip. The control unit (1010) may perform overall control operations of the interface unit (1000), the data processing unit (1020), and the storage unit (1030). The control unit (1010) may store in the storage unit (1030) model data related to the three-dimensional model, image data of the second image photographed by equipment such as the C-arm, and endoscopic image data, and perform data processing operations through the data processing unit (1020).

For example, the control unit (1010) may adjust the three-dimensional model of the patient's internal body structure generated by imaging, in cooperation with the data processing unit (1020), based on the image data of the two-dimensional image. The actual adjustment operation may be performed by the data processing unit (1020). Here, adjustment of the three-dimensional model refers to adjustment related to a change in the posture of the patient. For example, the three-dimensional model may be data generated in the first posture where the patient is lying supine. In contrast, the image data of the two-dimensional image may be data generated in the second posture (for example, prone posture or lateral posture) of the patient in the operating room for spinal surgery. Accordingly, the control unit (1010), in cooperation with the data processing unit (1020), may eliminate errors between the three-dimensional model and the actual state of the patient when the augmented reality device (120) is used for surgery, through adjustment of the three-dimensional model.

The data processing unit (1020), substantially under the control of the control unit (1010), and more specifically based on setting information configured on the window screen of the augmented reality device (120), may perform various operations for reducing errors between the three-dimensional model and the actual state of the patient. The data processing unit (1020) may also control so that the state of surgery performed in the operating room is implemented as real-time images on the augmented reality device (120). For example, the data processing unit (1020) may display the three-dimensional model on a virtual screen. At this time, by setting the display range of the three-dimensional model, only the three-dimensional model corresponding to the surgical range may be displayed.

The data processing unit (1020) may also perform an operation for setting reference points on the two-dimensional image data related to the actual surgical posture of the patient and on the three-dimensional model. A window for setting the reference points may be displayed on the augmented reality device (120), and the three-dimensional model may be adjusted based on the reference point information configured through the window. For example, the three-dimensional model may be adjusted by extracting 2D-3D matching points, extracting corresponding points in the three-dimensional model, performing 2D-3D point matching, applying a pose estimation algorithm to estimate posture, and performing operations such as skeletal rigging and surface deformation.

Furthermore, once the adjustment operation of the three-dimensional model has been completed, the data processing unit (1020) may perform an operation of setting a surgical instrument insertion path. Factors reflected in the computation process of the surgical instrument insertion path may include the positions of major neural tissues, major vascular tissues, and bone tissues, as well as the minimum insertion distance required for minimally invasive surgery. It is important to ensure that major nerves or organs are not damaged when the surgical instrument is inserted from the flank or from the back. In the case of endoscopic discectomy, it is important to derive the optimal surgical instrument insertion path (optimal position and angle) by considering the shape and position of the disc as the surgical target, as well as the shapes and positions of surrounding bone tissues, neural tissues, and vascular tissues. To this end, the data processing unit (1020) may execute a path-finding algorithm for path setting. Of course, in embodiments of the present invention, it is fully possible to apply artificial intelligence (AI) programs, and the invention is not limited to any particular form.

When the path has been set, the data processing unit (1020) may set the path on the three-dimensional model and provide related data so that the path is displayed on the screen of the augmented reality device (120). In addition, the data processing unit (1020), in cooperation with the control unit (1010), may process related data so that the three-dimensional model reflecting the surgical state through the path is implemented in real time on the augmented reality device (120).

The storage unit (1030) may temporarily store various types of data or information processed under the control of the control unit (1010). Here, since in practice the terms “data” and “information” are used interchangeably, the concepts of these terms are not particularly limited. However, data for image implementation may be referred to as pixel data, while information such as reference points set on the windows may be referred to as reference point information. The storage unit (1030) may temporarily store data of the three-dimensional model or image data photographed by X-ray equipment such as a C-arm, and then provide the data to the data processing unit (1020) for data processing.

In addition to the foregoing, the interface unit (1000), the control unit (1010), the data processing unit (1020), and the storage unit (1030) of FIG. 18 may perform various other operations, and further details have already been fully described above, which shall be incorporated herein by reference.

Meanwhile, the interface unit (1000), the control unit (1010), the data processing unit (1020), and the storage unit (1030) of FIG. 18 according to embodiments of the present invention are configured as hardware modules physically separated from one another, but each module may store and execute software for performing the above-described operations. However, since the software is a collection of software modules, and each module may fully be implemented in hardware, the invention is not particularly limited by whether the configuration is software or hardware. For example, the storage unit (1030) may be hardware such as storage or memory. However, storing information in software form, such as in a repository, is also fully possible, and the invention is not limited thereto.

In another embodiment of the present invention, the control unit (1010) may include a CPU and memory, and may be implemented as a single-chip structure. The CPU may include a control circuit, an arithmetic logic unit (ALU), an instruction interpreter, and registers, while the memory may include RAM. The control circuit may perform control operations, the arithmetic logic unit may perform operations on binary bit information, the instruction interpreter may include an interpreter or a compiler to convert high-level language into machine code and machine code into high-level language, and the registers may be involved in software-based data storage. According to this configuration, for example, at the initial operation of the data processing device (110) of FIG. 1, a program stored in the data processing unit (1020) may be copied into memory, that is, RAM, and executed, thereby rapidly increasing the speed of data processing. In the case of deep learning models, the program may be loaded into GPU memory instead of RAM and executed by utilizing the GPU to accelerate execution speed.

Meanwhile, although embodiments of the present invention have been described as comprising or operating with all components combined into one, the invention is not necessarily limited to such embodiments. That is, within the scope of the objectives of the present invention, all components may be selectively combined and operated as one or more. In addition, although all components may each be implemented as independent hardware, some or all of the components may alternatively be selectively combined and implemented as a computer program having program modules that perform some or all of the functions in one or more hardware devices. The codes and code segments constituting such a computer program may be readily derived by those skilled in the art to which the present invention pertains. Such a computer program may be stored on non-transitory computer-readable media and read and executed by a computer, thereby implementing embodiments of the present invention.

Here, the term “non-transitory computer-readable media” refers to media that semi-permanently store data and can be read by a device, as opposed to media that temporarily store data for only a short time, such as registers, caches, or memories. Specifically, the above-described programs may be stored and provided on non-transitory computer-readable media such as CDs, DVDs, hard disks, Blu-ray discs, USBs, memory cards, or ROMs.

As described above, the present invention has been explained by specific components, limited embodiments, and drawings. However, these are provided merely for better overall understanding of the present invention, and the present invention is not limited to the above embodiments. Those skilled in the art to which the present invention pertains may make various modifications and variations based on the disclosures herein without departing from the scope of the present invention.

### DESCRIPTION OF REFERENCE NUMERALS

101: First medical imaging device
103: Second medical imaging device
105: Third medical imaging device
110, 110′: Data processing device
120: Augmented reality device
130: Interface system
100: Medical imaging acquisition system
140: Position and environment recognition system
150: Image projection system
160: Interface system

1. A spinal surgery image-providing method comprising:
storing a three-dimensional model generated using first image data acquired by a first medical imaging device when a patient is in a first posture;
storing second image data acquired by a second medical imaging device when the patient is in a second posture;
adjusting the three-dimensional model using the second image data in a three-dimensional model adjustment step;
computing an optimal surgical instrument insertion path toward a surgical target in an insertion path computation step; and
displaying the optimal surgical instrument insertion path on a screen of an augmented reality device or on a body surface of the patient in an insertion path display step.

2. The method of claim 1, wherein the three-dimensional model adjustment step comprises:
extracting feature points from the second image data;
extracting corresponding points from the three-dimensional model that correspond to the feature points;
mapping correspondences between the feature points and the corresponding points;
estimating the second posture using the mapped correspondences; and
adjusting the three-dimensional model according to the estimated second posture.

3. The method of claim 1, wherein the three-dimensional model comprises one or more layers, the layers including at least one selected from the group consisting of a skin layer, a bone layer, an intervertebral disc layer, a ligament layer, a nerve layer, and a vascular layer, and
wherein the method further comprises a display range setting step of setting a display range of the three-dimensional model displayed on the screen of the augmented reality device or on the body surface of the patient, the display range setting step comprising:
displaying, on the screen of the augmented reality device or on an interface system, a first list in which predetermined anatomical structures are enumerated and receiving a selection value input by a user; or
displaying, on the screen of the augmented reality device or on the interface system, the three-dimensional model and receiving a selection value input by the user; or
displaying, on the screen of the augmented reality device or on the interface system, a second list in which the layers are enumerated and receiving a selection value input by the user.

4. The method of claim 1, wherein the first medical imaging device is a CT (computed tomography) device or an MRI (magnetic resonance imaging) device,
wherein the three-dimensional model comprises at least one selected from the group consisting of a skin layer, a bone layer, an intervertebral disc layer, a ligament layer, a nerve layer, and a vascular layer, and
wherein the second medical imaging device is a C-arm device.

5. The method of claim 1, wherein the three-dimensional model adjustment step comprises:
displaying the three-dimensional model on a screen of the augmented reality device or on an interface system, and receiving a first reference point input by a user with respect to a characteristic structure of the three-dimensional model in a first reference point reception step;
visually displaying the second image data on the screen of the augmented reality device or on the interface system, and receiving a second reference point input by a user with respect to a characteristic structure of the second image data in a second reference point reception step; and
adjusting the three-dimensional model using the first reference point and the second reference point in a fine adjustment step.

6. The method of claim 5, wherein the characteristic structure comprises at least one of a tip of a spinous process or an iliac crest.

7. The method of claim 5, wherein the second reference point reception step comprises:
visually displaying, on the screen of the augmented reality device or on the interface system, second-1 image data photographed in a direction viewing the patient from above downward, and receiving second-1 reference points input by a user with respect to characteristic structures of the second-1 image data; and
visually displaying, on the screen of the augmented reality device or on the interface system, second-2 image data photographed in a direction viewing the flank of the patient, and receiving a second-2 reference point input by a user with respect to characteristic structures of the second-2 image data.

8. The method of claim 5, wherein the fine adjustment step comprises:
mapping correspondences between the first reference point and the second reference point;
estimating the second posture based on the mapped correspondences; and
adjusting the three-dimensional model according to the estimated second posture.

9. The method of claim 1, wherein the insertion path computation step further comprises a surgical target setting step of setting a surgical target with respect to the three-dimensional model, the surgical target setting step comprising:
invoking a surgical target setting menu through the augmented reality device or the interface system, and storing, as a surgical target model, an item selected by a user from the surgical target setting menu; or
storing, as a surgical target model, a region designated by a user with respect to the three-dimensional model displayed on the augmented reality device or on the interface system.

10. The method of claim 1, wherein the insertion path computation step comprises:
dividing the three-dimensional model into a plurality of small voxels;
storing an initial position where a surgical instrument is to be inserted and a surgical target position where the surgical instrument is to reach;
assigning movement costs to each voxel in a cost assignment step; and
computing costs required to pass through voxels between the initial position and the surgical target position and determining, as the optimal surgical instrument insertion path, a path having the lowest cost.

11. The method of claim 10, wherein the cost assignment step comprises assigning relatively high costs to voxels adjacent to predetermined anatomical structures, or assigning relatively high costs to voxels located at positions into which the surgical instrument cannot be inserted or is difficult to insert.

12. The method of claim 1, further comprising a surgical instrument model display step of displaying a surgical instrument model representing a surgical instrument in three dimensions on the screen of the augmented reality device or on the body surface of the patient.

13. The method of claim 12, wherein the surgical instrument model display step comprises:
detecting information regarding the surgical instrument from the second image data or receiving information regarding the surgical instrument; and
displaying the surgical instrument model based on the information regarding the surgical instrument, overlapped with the three-dimensional model.

14. The method of claim 12, wherein the optimal surgical instrument insertion path and the surgical instrument model are displayed overlapped together on the screen of the augmented reality device or on the body surface of the patient.

15. The method of claim 1, further comprising, after the insertion path display step, visually displaying third image data acquired by a third medical imaging device on the screen of the augmented reality device or on the interface system.

16. The method of claim 15, wherein the third medical imaging device is an endoscopic camera.

17. The method of claim 1, further comprising a surgical state reflection step of displaying the three-dimensional model reflecting a surgical state on at least one of the screen of the augmented reality device, the body surface of the patient, or the interface system,
wherein the surgical state reflection step comprises computing surgical progress using at least one of the number of times the surgical instrument model passes through each position, the three-dimensional range in which the surgical instrument model has moved, and the residence time of the surgical instrument model at each position, and reflecting on the surgical target model the region in which surgery has been performed according to the surgical progress.

18. The method of claim 17, wherein the surgical state reflection step further comprises:
displaying the surgical target model in different colors according to the surgical progress, or displaying the surgical target model with different transparency according to the surgical progress; and
providing a notification to a user through the augmented reality device when the surgical instrument model approaches within a predetermined distance of a predetermined anatomical structure or when the surgical progress reaches a preset level.

19. A non-transitory computer-readable medium storing a program for executing the spinal surgery image-providing method according to claim 1.
